SEQUENCE LISTING (23 sequences)

SEQ ID NO: 1 - DNA, 248 bp, Artificial Sequence
  Synthetic polynucleotide sequence.
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 2 - DNA, 383 bp, Artificial Sequence
  Synthetic polynucleotide sequence.
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 3 - DNA, 282 bp, Artificial Sequence
  Synthetic polynucleotide sequence.
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 4 - DNA, 444 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 5 - DNA, 145 bp, Artificial Sequence
  Synthetic polynucleotide sequence.
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - DNA, 145 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 130 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 119 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggcggcctcagtgagcgagcgagcgcgcag

SEQ ID NO: 9 - DNA, 20 bp, Artificial Sequence
  Synthetic polynucleotide sequence.
cacgttgaccagcatggtgt

SEQ ID NO: 10 - DNA, 20 bp, Artificial Sequence
  Synthetic polynucleotide sequence.
gacgtgtccaagaaattgat

SEQ ID NO: 11 - DNA, 12 bp, Artificial Sequence
  Synthetic polynucleotide sequence.
  misc_feature (5)..(5): n can be any nucleotide.
  misc_feature (6)..(6): n can be any nucleotide.
  misc_feature (7)..(7): n can be any nucleotide.
  misc_feature (8)..(8): n can be any nucleotide.
gatcnnnngatc

SEQ ID NO: 12 - DNA, 153 bp, Artificial Sequence
  HBoV1 WT ITR 5′
ttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgc

SEQ ID NO: 13 - DNA, 150 bp, Artificial Sequence
  HBoV1 WT ITR 3′
tatatgtgacgtggttgtacagacgccatcttggaatccaatatgtctgccggcgattagatcatgcgcgcgcgcagcgcgctgcgcgcagcgcaggcatgactgagccggcagacatattggattccaagatggcgtctgtacaaccac

SEQ ID NO: 14 - DNA, 7891 bp, Artificial Sequence
  V2.0 Human codon optimized mTTR-Intron-BDD-FVIIIXTEN-WPRE-bGHPoly expression cassette
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaag

SEQ ID NO: 15 - DNA, 4824 bp, Artificial Sequence
  Nucleotide sequence encoding B-domain deleted (BDD) codon-optimized human Factor VIII (BDDcoFVIII) fused with 144 amino acid XTEN
atgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 16 - DNA, 330 bp, Artificial Sequence
  A1MB2 ENHANCER
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccacc

SEQ ID NO: 17 - DNA, 345 bp, Artificial Sequence
  mTTR promoter
gatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctag

SEQ ID NO: 18 - DNA, 1489 bp, Artificial Sequence
  CHIMERIC INTRON
tcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaatta

SEQ ID NO: 19 - DNA, 595 bp, Artificial Sequence
  WPRE
tcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctg

SEQ ID NO: 20 - DNA, 211 bp, Artificial Sequence
  bGHpA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 21 - DNA, 4317 bp, Artificial Sequence
  Nucleotide sequence of coFVIII-6
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 22 - DNA, 4335 bp, Artificial Sequence
  Nuc_encoding_BDDcodon-optimized human Factor VII_no XTEN
gccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccggcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 23 - DNA, 4824 bp, Artificial Sequence
  V1.0expression cassette
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

(19) United States
(12) Patent Application Publication
(10) Pub. No.: US 2022/0090130 A1
(43) Pub. Date: Mar. 24, 2022

(54) MODIFIED BACULOVIRUS SYSTEM FOR IMPROVED PRODUCTION OF CLOSED-ENDED DNA (ceDNA)

(71) Applicant: BIOVERATIV THERAPEUTICS INC., Waltham, MA (US)

(72) Inventor: Ajay Maghodia, Waltham, MA (US)

(21) Appl. No.: 17/409,774

(22) Filed: Aug. 23, 2021

Related U.S. Application Data
(60) Provisional application No. 63/069,115, filed on Aug. 23, 2020.

(51) Int. Cl. C12N 15/86; C07K 14/755

(52) U.S. Cl. CPC C12N 15/86; C07K 14/755; C12N 2710/14052; C12N 2820/55; C12N 2710/14043

(57) ABSTRACT

The present disclosure relates to a recombinant baculovirus expression vector (rBEV) for the production of closed-ended DNA (ceDNA) in insect cells.

### RELATED APPLICATIONS

This application claims priority to U.S. Provisional Patent Application No. 63/069,115, filed Aug. 23, 2020, the disclosure of which is hereby incorporated by reference in its entirety.

### REFERENCE TO SEQUENCE LISTING SUBMITTED ELECTRONICALLY

The content of the electronically submitted sequence listing in ASCII text file (Name: 720560_SA9-475_ST25.txt; Size: 44.3 kB; and Date of Creation: Aug. 23, 2021) is incorporated herein by reference in its entirety.

### BACKGROUND

Gene therapy offers a lasting means of treating a variety of diseases. In the past, gene therapy typically relied on the use of viral vectors. AAV vectors have emerged as one of the more common types of viral vectors. However, the presence of the capsid limits the utility of an AAV vector in gene therapy. In particular, the capsid itself can limit the size of the transgene that is included in the vector to as low as less than 4.5 kb. Various therapeutic proteins that may be useful in a gene therapy can easily exceed this size even before expression control sequences are added. Furthermore, proteins that make up the capsid can serve as antigens that can be targeted by a subject's immune system. AAV is very common in the general population, with most people having been exposed to an AAV throughout their lives. As a result, most potential gene therapy recipients have likely already developed an immune response to an AAV, and thus are more likely to reject the therapy. Moreover, viral vector production in mammalian cells may suffer from low yields and the difficulty in scaling up for large-scale commercial production.

It has been shown that in the absence of AAV cap gene expression, an AAV vector genome undergoes inefficient replication and the complementary strands of the intramolecular intermediate, covalently linked through the ITRs on both ends, accumulates in a novel conformation of closed-ended linear duplex DNA (ceDNA). ceDNA do not have packaging constraints imposed by the limiting space within a viral capsid. Accordingly, ceDNA vectors may be used as an alternative to viral vector gene therapy. Control elements, large transgenes, and multiple transgenes may be included in a ceDNA construct without concern for size limit.

A baculovirus expression vector (BEV) is a recombinant baculovirus with a double-stranded circular DNA genome that has been genetically modified to include a foreign gene of interest. BEVs are viable and can infect susceptible hosts, usually cultured insect cells. BEVs can be used to produce closed-ended DNA (ceDNA) for gene therapy and thereby avoid the need for viral vector. However, it has been found that when a nucleic acid of interest, such as ceDNA, is purified from the insect cells after being transduced with a BEV, baculovirus genomic DNA can be found to be co-purified along with the nucleic acid of interest. This seems to be due to viral particles that are produced.

Thus, there exists a need in the art to efficiently produce a purified nucleic acid of interest in a baculovirus system, and reduce the number of progeny virus particles, and ultimately, the contamination of baculoviral genomic DNA in purified nucleic acid preparations.

### SUMMARY OF THE DISCLOSURE

The present disclosure is directed, at least in part, to an expression system comprising (1) a recombinant bacmid or recombinant baculovirus expression vector (rBEV) comprising an edited genome with an inactivated or attenuated baculovirus gene that is essential for baculovirus replication (e.g., an inactivated capsid gene, e.g., an inactivated VP80 gene) and a nucleic acid of interest (e.g., a ceDNA vector); and (2) a functional counterpart of the inactivated or attenuated essential baculovirus gene that is provided in trans, such that a host cell (e.g., insect cell) is capable of propagating the rBEV following infection of the host cells with the rBV.

It has been discovered that the expression system of the disclosure enables the production of a nucleic acid of interest (e.g., ceDNA) without appreciable levels of contaminating BV genomic DNA. Therefore, DNA isolated from host cells (e.g., insect cells) infected with genome-edited rBEV produce higher titers of DNA than host cells infected with rBV having a genome containing a functional counterpart of the essential baculovirus gene. These discoveries have been exploited to develop the present disclosure, which, in part, is directed to a recombinant baculovirus system, the components thereof, and to methods using a specifically edited rBV for production of heterologous DNA (e.g., ceDNA).

In one aspect, the disclosure provides a recombinant bacmid, comprising: (i) a variant of a baculovirus gene required for baculovirus replication, wherein the variant gene exhibits reduced expression of its encoded protein; (ii) a bacterial origin of replication (ori); and (iii) at least one integration site for integration of a heterologous DNA sequence comprising a transgene.

In one embodiment, the baculovirus gene is a capsid or capsid-associated gene.

In one embodiment, the baculovirus gene is selected from the group consisting of VP80, VP39, GP41, P333, VP1-54, VLF-1, and PP78/83.

In one embodiment, the baculovirus gene is VP80.

In one embodiment, the variant of the essential gene is not expressed due to a disruption or mutation that inactivates its expression.

In one embodiment, the variant of the essential gene comprises an insertion and/or deletion (“indel”) that disrupts its expression.

In one embodiment, the indel is generated by a targeted nuclease system.

In one embodiment, the origin of replication is a mini-F-replicon, ColE1, oriC, OriV, OriT or OriS.

In one embodiment, the bacmid further comprises a reporter gene.

In one embodiment, the bacmid further comprises a selection marker expression gene cassette.

In one embodiment, the bacmid further comprises a Rep protein.

In another aspect, the disclosure provides recombinant baculovirus expression vector (rBEV) generated by site specific integration of a heterologous DNA sequence into the integration site of the bacmid of any one of the preceding claims.

In one embodiment, the heterologous DNA sequence is a Rep protein.

In another embodiment, the heterologous nucleic acid sequence comprises a transgene flanked by Inverted Terminal Repeats (ITRs).

In one embodiment, the heterologous nucleic acid is expressed as closed ended DNA (ceDNA).

In another aspect, the disclosure provides a baculovirus expression system comprising (i) an rBEV as disclosed herein; and (ii) a source of functional protein wherein the functional protein is capable of complementing the variant essential gene and wherein the functional protein is provided in trans to the rBEV.

In one embodiment, the functional protein is provided as a separate expression vector which expresses the functional protein in trans.

In one embodiment, the functional protein is provided by an insect cell which expresses functional capsid protein corresponding to the variant capsid protein.

In one embodiment, the insect cell is a Sf9, Sf21, S2, Trichoplusia ni, E4a, or BTI-TN-5B1-4 cell.

In another embodiment, the insect cell is a stable cell line that encodes a heterologous nucleic acid sequence.

In another embodiment, heterologous DNA sequence comprises the transgene flanked by Inverted Terminal Repeats (ITRs).

In another embodiment, the heterologous nucleic acid is expressed as closed ended DNA (ceDNA).

In another aspect, the disclosure provides a method of propagating a baculovirus expression vector in an insect cell the method comprising: (a) transfecting the insect cell with a recombinant baculovirus expression vector (rBEV) as disclosed herein; (b) providing a functional protein capable of complementing the variant essential gene wherein the functional protein is provided in trans to the rBEV; and (c) culturing the insect cell, thereby propagating the baculovirus expression system vector.

In one embodiment, the functional protein is provided by electroporating the insect cell with the functional capsid protein.

In one embodiment, the functional protein is provided by transfecting the insect cell with a separate expression vector which stably integrates and expresses the functional protein in trans.

In one embodiment, the functional capsid gene is provided by expressing the functional capsid protein corresponding to the variant capsid protein in the insect cell.

In one embodiment, the functional capsid gene is expressed in the cell under the control of an inducible or transactivating promoter.

In one embodiment, the inducible promoter is the Autographa californica nucleopolyhedrovirus (AcMNPV) 39K promoter.

In another aspect, the disclosure provides a method of producing a heterologous DNA sequence comprising a transgene, (a) propagating a recombinant baculovirus expression vector (rBEV) as disclosed herein; (b) harvesting the rBEV; (c) infecting a stable insect cell insect cell with the harvested rBEV, wherein the stable insect cell line encodes a heterologous nucleic acid sequence; and (d) purifying the heterologous DNA sequence expressed in the stable insect cell line.

In another aspect, the disclosure provides a method of producing a heterologous DNA sequence comprising a transgene, (a) propagating a recombinant baculovirus expression vector (rBEV) as disclosed herein; (b) harvesting the rBEV; (c) infecting an insect cell to express a heterologous DNA sequence; and (d) purifying the heterologous DNA sequence from the insect cell.

In one embodiment, the heterologous DNA sequence is substantially free of baculovirus genomic DNA.

In one embodiment, the heterologous DNA sequence comprises the transgene flanked by Inverted Terminal Repeats (ITRs).

In one embodiment, the heterologous nucleic acid is expressed as closed ended DNA (ceDNA).

In another aspect, the disclosure provides a heterologous DNA sequence comprising a transgene encoding a therapeutic protein, the heterologous DNA sequence produced by the methods disclosed herein.

The foregoing and other objects of the present disclosure, the various features thereof, as well as the disclosure itself may be more fully understood from the following description, when read together with the accompanying drawings.

### BRIEF DESCRIPTION OF THE DRAWINGS

FIG. 1 depicts schematic map of a recombinant baculovirus expression vector encoding AAV2.Rep (AcBIWBac.Polh.AAV2.Rep^{Tn7}).

FIG. 2 illustrates where two single-guide RNAs (sgRNAs) target the VP80 gene locus within the baculovirus expression vector.

FIG. 3 and FIG. 4 illustrate the TIDE (Tracking of Indels by Decomposition) analysis of two separate clones to determine the indels induced by each sgRNA.

FIG. 5A is an illustration of the transfer vector encoding the AcMNPV vp80 gene under the inducible AcMNPV 39K promoter used for the generation of a Sf.39K.VP80 complement cell line. FIG. 5B shows a schematic map of a plasmid encoding neomycin resistance marker under the AcMNPV immediate early (ie1) promoter preceded by the transcriptional enhancer hr5 element and followed by the AcMNPV p10 polyadenylation signal. FIG. 5C shows a schematic map of a hFVIIIco6XTEN expression cassette flanked by AAV2 ITRs, which is stably integrated into the Sf9 cell genome to generate a stable cell line.

FIG. 6 is a gel assay showing a single thick band of the hFVIIIco6XTEN closed-ended DNA (ceDNA) produced by the modified rBEV in comparison with its unmodified counterpart.

FIG. 7 shows the agarose gel image of the hFVIIIco6XTEN ceDNA analyzed before (uncut) and after (right side of the marker) the restriction enzyme digestion as described in Example 5. Heat-treated samples ran at different volumes are indicated under the “heat-treated” lanes and untreated samples ran at different volumes are indicated under the “untreated” lanes. DNA size fragments obtained according to the map described in FIG. 8 are indicated on the right with arrows and sizes in kb.

FIG. 8 shows the schematic map of the AscI restriction endonuclease digestion of the hFVIIIco6XTEN ceDNA. AscI has a single recognition site in the hFVIIIco6XTEN monomer of 6556 bp in size and generates 2.9 kb and 3.6 kb fragments after the digestion. Red rectangles indicate the position of 5′ ITRs and black rectangles indicate the position of 3′ ITRs. Schematic maps of two dimer figures in tail-to-tail or head-to-head conformations are also shown along with the AscI recognition site(s) and predicted DNA fragments sizes indicated in kb.

### DETAILED DESCRIPTION

The present disclosure describes the downregulation of expression of a capsid gene from the baculovirus genome to prevent contamination in heterologous DNA preparations for gene therapy purposes.

### Definitions

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

It is to be noted that the term “a” or “an” entity refers to one or more of that entity: for example, “a nucleotide sequence” is understood to represent one or more nucleotide sequences. Similarly, “a therapeutic protein” and “a baculovirus expression vector” is understood to represent one or more therapeutic protein and one or more baculovirus expression vector, respectively. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Also as used herein, “and/or” refers to and encompasses any and all possible combinations of one or more of the associated listed items, as well as the lack of combinations when interpreted in the alternative (“or”).

“Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, the term “heterologous nucleotide sequence” refers to a nucleotide sequence that does not naturally occur with a given polynucleotide sequence. In certain embodiments, the heterologous nucleotide sequence comprises a transgene.

As used herein, the term “transgene” refers to a nucleic acid of interest (other than a nucleic acid encoding a capsid polypeptide) that is incorporated into and may be delivered and expressed by a nucleic acid molecule, e.g., a ceDNA vector, as disclosed herein. Transgenes of interest include, but are not limited to, nucleic acids encoding polypeptides, preferably therapeutic (e.g., for medical, diagnostic, or veterinary uses) or immunogenic polypeptides (e.g., for vaccines). In some embodiments, nucleic acids of interest include nucleic acids that are transcribed into therapeutic RNA. Transgenes included for use in the nucleic acid molecules of the disclosure (e.g., ceDNA vectors) include, but are not limited to, those that express or encode one or more polypeptides, peptides, ribozymes, aptamers, peptide nucleic acids, siRNAs, RNAis, miRNAs, IncRNAs, antisense oligo- or polynucleotides, antibodies, antigen binding fragments, or any combination thereof.

As used herein, an “inverted terminal repeat” (or “ITR”) refers to a nucleic acid subsequence located at either the 5′ or 3′ end of a single stranded nucleic acid sequence (e.g., an expression cassette or transgene), which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC; SEQ ID NO:11) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop. In some embodiments, the ITR forms a T-shaped hairpin structure. In some embodiments, the ITR forms a non-T-shaped hairpin structure, e.g., a U-shaped hairpin structure.

An ITR can have any number of functions. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell. In the context of a virus, ITRs mediate replication, virus packaging, integration and provirus rescue. In the context of a nucleic acid molecule (e.g., a ceDNA vector) devoid of capsid genes and flanked by ITR sequences, the ITR is capable of mediating replication of the nucleic acid molecule (e.g., ceDNA vector).

In certain embodiments, the ITR is viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) may together constitute a “minimal required origin of replication”.

It will be understood that more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19). Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species.

In certain embodiments, at least one ITR is an ITR of a non-adenovirus associated virus (non-AAV). In certain embodiments, the ITR is an ITR of a non-AAV member of the viral family Parvoviridae. In some embodiments, the ITR is an ITR of a non-AAV member of the genus Dependovirus or the genus Erythrovirus. In particular embodiments, the ITR is an ITR of a goose parvovirus (GPV), a Muscovy duck parvovirus (MDPV), or an erythrovirus parvovirus B19 (also known as parvovirus B19, primate erythroparvovirus 1, B19 virus, and erythrovirus). In certain embodiments, one ITR of two ITRs is an ITR of an AAV. In other embodiments, one ITR of two ITRs in the construct is an ITR of an AAV serotype selected from serotype 1, 2, 3, 4, 5, 6, 7, 8, 9, 10, 11 and any combination thereof. In one particular embodiment, the ITR is derived from AAV serotype 2, e.g., an ITR of AAV serotype 2.

In certain embodiments, the ITR can be further be modified by truncation, substitution, deletion, insertion and/or addition. In one embodiment, the initial sequence and/or the reverse complement comprise about 2-600 nucleotides, about 2-550 nucleotides, about 2-500 nucleotides, about 2-450 nucleotides, about 2-400 nucleotides, about 2-350 nucleotides, about 2-300 nucleotides, or about 2-250 nucleotides. In some embodiments, the initial sequence and/or the reverse complement comprise about 5-600 nucleotides, about 10-600 nucleotides, about 15-600 nucleotides, about 20-600 nucleotides, about 25-600 nucleotides, about 30-600 nucleotides, about 35-600 nucleotides, about 40-600 nucleotides, about 45-600 nucleotides, about 50-600 nucleotides, about 60-600 nucleotides, about 70-600 nucleotides, about 80-600 nucleotides, about 90-600 nucleotides, about 100-600 nucleotides, about 150-600 nucleotides, about 200-600 nucleotides, about 300-600 nucleotides, about 350-600 nucleotides, about 400-600 nucleotides, about 450-600 nucleotides, about 500-600 nucleotides, or about 550-600 nucleotides. In some embodiments, the initial sequence and/or the reverse complement comprise about 5-550 nucleotides, about 5 to 500 nucleotides, about 5-450 nucleotides, about 5 to 400 nucleotides, about 5-350 nucleotides, about 5 to 300 nucleotides, or about 5-250 nucleotides. In some embodiments, the initial sequence and/or the reverse complement comprise about 10-550 nucleotides, about 15-500 nucleotides, about 20-450 nucleotides, about 25-400 nucleotides, about 30-350 nucleotides, about 35-300 nucleotides, or about 40-250 nucleotides. In certain embodiments, the initial sequence and/or the reverse complement comprise about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, or about 600 nucleotides. In particular embodiments, the initial sequence and/or the reverse complement comprise about 400 nucleotides.

In other embodiments, the initial sequence and/or the reverse complement comprise about 2-200 nucleotides, about 5-200 nucleotides, about 10-200 nucleotides, about 20-200 nucleotides, about 30-200 nucleotides, about 40-200 nucleotides, about 50-200 nucleotides, about 60-200 nucleotides, about 70-200 nucleotides, about 80-200 nucleotides, about 90-200 nucleotides, about 100-200 nucleotides, about 125-200 nucleotides, about 150-200 nucleotides, or about 175-200 nucleotides. In other embodiments, the initial sequence and/or the reverse complement comprise about 2-150 nucleotides, about 5-150 nucleotides, about 10-150 nucleotides, about 20-150 nucleotides, about 30-150 nucleotides, about 40-150 nucleotides, about 50-150 nucleotides, about 75-150 nucleotides, about 100-150 nucleotides, or about 125-150 nucleotides. In other embodiments, the initial sequence and/or the reverse complement comprise about 2-100 nucleotides, about 5-100 nucleotides, about 10-100 nucleotides, about 20-100 nucleotides, about 30-100 nucleotides, about 40-100 nucleotides, about 50-100 nucleotides, or about 75-100 nucleotides. In other embodiments, the initial sequence and/or the reverse complement comprise about 2-50 nucleotides, about 10-50 nucleotides, about 20-50 nucleotides, about 30-50 nucleotides, about 40-50 nucleotides, about 3-30 nucleotides, about 4-20 nucleotides, or about 5-10 nucleotides. In another embodiment, the initial sequence and/or the reverse complement consist of two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In other embodiments, an intervening nucleotide between the initial sequence and the reverse complement is (e.g., consists of) 0 nucleotide, 1 nucleotide, two nucleotides, three nucleotides, four nucleotides, five nucleotides, six nucleotides, seven nucleotides, eight nucleotides, nine nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

In certain aspects of the present disclosure, the nucleic acid molecule comprises two ITRs, a 5′ ITR and a 3′ ITR, wherein the 5′ ITR is located at the 5′ terminus of the nucleic acid molecule, and the 3′ ITR is located at the 3′ terminus of the nucleic acid molecule. The 5′ ITR and the 3′ ITR can be derived from the same virus or different viruses. In certain embodiments, the 5′ ITR is derived from an AAV and the 3′ ITR is not derived from an AAV virus (e.g., a non-AAV). In some embodiments, the 3′ ITR is derived from an AAV and the 5′ ITR is not derived from an AAV virus (e.g., a non-AAV). In other embodiments, the 5′ ITR is not derived from an AAV virus (e.g., a non-AAV), and the 3′ ITR is derived from the same or a different non-AAV virus.

In certain embodiments, the pair of ITRs are asymmetric ITRs. As used herein, the term “asymmetric ITRs” refers to a pair of ITRs that are not inverse complements across their full length. The difference in sequence between the two ITRs may be due to nucleotide addition, deletion, truncation, or point mutation. In one embodiment, one ITR of the pair may be a wild-type AAV or non-AAV sequence and the other a non-wild-type or synthetic sequence. In another embodiment, neither ITR of the pair is a wild-type sequence and the two ITRs differ in sequence from one another. For convenience herein, an ITR located 5′ to (upstream of) an expression cassette may be referred to as a “5′ ITR” or a “left ITR”, and an ITR located 3′ to (downstream of) an expression cassette may be referred to as a “3′ ITR” or a “right ITR”.

As used herein, the terms “Rep binding site,” Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. Any known RBS sequence may be used in the embodiments of the invention, including naturally known or synthetic RBS sequences. Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the term “genetic cassette” or “expression cassette” means a DNA sequence capable of directing expression of a particular polynucleotide sequence in an appropriate host cell, comprising a promoter operably linked to a polynucleotide sequence of interest. A genetic cassette may encompass nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence. In some embodiments, the genetic cassette comprises a polynucleotide which encodes a gene product. In some embodiments, the genetic cassette comprises a polynucleotide which encodes a miRNA. In some embodiments, the genetic cassette comprises a heterologous polynucleotide sequence.

A polynucleotide which encodes a product, e.g., a miRNA or a gene product (e.g., a polypeptide such as a therapeutic protein), can include a promoter and/or other expression (e.g., transcription or translation) control sequences operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control sequences, besides a promoter, for example enhancers, operators, repressors, and transcription termination signals, can also be operably associated with a coding region to direct gene product expression.

“Expression control sequences” refer to regulatory nucleotide sequences, such as promoters, enhancers, terminators, and the like, that provide for the expression of a coding sequence in a host cell. Expression control sequences generally encompass any regulatory nucleotide sequence which facilitates the efficient transcription and translation of the coding nucleic acid to which it is operably linked. Non-limiting examples of expression control sequences include promoters, enhancers, translation leader sequences, introns, polyadenylation recognition sequences, RNA processing sites, effector binding sites, or stem-loop structures. A variety of expression control sequences are known to those skilled in the art. These include, without limitation, expression control sequences which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other expression control sequences include those derived from vertebrate genes such as actin, heat shock protein, bovine growth hormone and rabbit β-globin, as well as other sequences capable of controlling gene expression in eukaryotic cells. Additional suitable expression control sequences include tissue-specific promoters and enhancers as well as lymphokine-inducible promoters (e.g., promoters inducible by interferons or interleukins). Other expression control sequences include intronic sequences, post-transcriptional regulatory elements, and polyadenylation signals. Additional exemplary expression control sequences are discussed elsewhere in the present disclosure.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), red fluorescent protein (RFP), chloramphenicol acetyltransferase (CAT), β-galactosidase (LacZ), β-glucuronidase (Gus), and the like. Selectable markers can also be considered to be reporters.

As used herein, the terms “closed-ended DNA vector”, “ceDNA vector” and “ceDNA” are used interchangeably and refer to a non-virus capsid-free DNA vector with at least one covalently-closed end (i.e., an intramolecular duplex). In some embodiments, the ceDNA comprises two covalently-closed ends. In certain embodiments, the ceDNA is produced from a template DNA or expression cassette that further incorporates at least one ITR. In certain embodiment, the ceDNA is incorporated as an intermolecular duplex polynucleotide of DNA in a baculovirus expression vector described herein. ceDNA vectors may be distinguished from plasmid-based expression vectors in a number of ways. For example, ceDNA vectors may possess one or more of the following features: (1) the lack of original (i.e. not inserted) bacterial DNA, (2) the lack of a prokaryotic origin of replication, (3) being self-containing, i.e., they do not require any sequences other than ITRs, (4) the presence of ITR sequences that form hairpins, (5) they are eukaryotic origin (i.e., they are produced in eukaryotic cells), and (6) the absence of bacterial-type DNA methylation. Another important feature distinguishing ceDNA vectors from plasmid expression vectors is that ceDNA vectors are single-strand linear DNA having closed ends, while plasmids are always double-stranded DNA. In certain embodiments, ceDNA vectors have a linear and continuous structure rather than a non-continuous structure, as determined by restriction enzyme digestion assay. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In certain embodiment, a ceDNA vector is resistant to exonuclease digestion (e.g. exonuclease I or exonuclease III), e.g. for over an hour at 37° C., due to the presence of one or more covalently closed ends.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Culture,” “to culture,” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology but is not necessarily translated from a designated nucleic acid sequence. It can be generated in any manner, including by chemical synthesis.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein, the term “therapeutic protein” refers to any polypeptide known in the art that can be administered to a subject. In some embodiments, the therapeutic protein comprises a protein selected from a clotting factor, a growth factor, an antibody, a functional fragment thereof, or a combination thereof.

As used herein, the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “gene editing” refers to a polynucleotide or a nucleic acid that has been edited to modify expression of the said polynucleotide or nucleic acid. The polynucleotide or nucleic acid may encode a protein. The gene editing may be targeted to a particular gene or locus of a genome or a heterologous nucleic acid, such as a baculovirus expression vector.

The term “bacmid” refers to a shuttle vector that can be propagated in both E. coli and insect cells. A genome-edited bacmid is a bacmid having an inactivated or attenuated capsid gene.

### Genome-Edited Bacmid

In certain aspects, the present disclosure provides a variant recombinant bacmid that is incapable of replication, or exhibits reduced replication, due to attenuated or inactivated expression of baculovirus gene that is essential for baculovirus (BV) replication. For example, a recombinant bacmid of the disclosure comprises portions of a WT baculovirus genome but deficient for at least one baculovirus gene essential baculovirus replication. The gene essential for BV replications may be either absent from the genome or its expression may be prevented or attenuated. In certain embodiments, the gene is mutated, e.g., by deletion or truncated, or otherwise inactivated.

In certain embodiments, a recombinant bacmid of the disclosure comprises DNA backbone wherein at least one baculovirus gene required for replication of a baculovirus is inactivated or attenuated by genome editing. In other embodiments, the baculovirus gene is reduced by an expression control system provided on the bacmid or in the host cell (e.g., insect cell) to be infected by the baculovirus.

Any genome derived from a baculovirus commonly used for the recombinant expression of proteins and biopharmaceutical products may be genome edited. For example, the baculovirus genome may be derived from for instance AcMNPV, BmNPV, Helicoverpa armigera (HearNPV) or Spodoptera exigua MNPV, preferably from AcMNPV or BmNPV. In particular, the baculovirus genome may be derived from the AcMNPV clone C6 (genomic sequence: Genbank accession no. NC_001623.1). In certain embodiments, a genome-edited backbone can be created from a bacmid comprising the WT baculovirus genome (AcMNPV (NC_001623) by editing an essential gene required for baculovirus replicaton in the WT baculovirus genome.

In certain embodiments, the baculovirus gene is a gene that is essential for baculovirus virion assembly. In certain embodiments, deficiency or inactivation of the gene negatively impacts the BV virions produced from a BV-infected cell. In certain embodiments, the bacmid of the disclosure comprises an inactivated or attenuated baculovirus gene encoding any of the following proteins: Ac100 (P6.9 DNA binding protein); AC89 (VP39 capsid); Ac80 (Gp41 tegument), Ac142, Ac144, Ac 66, Ac92 (P33), p6.9, Ac54 (VP1054), Ac77 (VLF-1), Ac104 (VP80), and Ac9 (PP78/83). In certain exemplary embodiments, the baculovirus gene to be targeted is VP 80.

In certain embodiments, the baculovirus gene may be inactivated by introducing a modification of said gene that results in the complete absence of a functional essential gene product. Accordingly, said mutation may result in the introduction of one or several stop codons in the open reading frame of the mRNA transcribed from the essential gene or may correspond to the deletion, either total or partial, of the essential gene. Alternatively, the gene may be mutated by way of nucleotide substitution, insertion or deletion in the sequence of all or a part of the wild type gene. The mutation may correspond to the complete deletion of the gene, or to only a part of said gene. For example, one may delete at least 50%, at least 60%, at least 70%, at least 80% or at least 90% of the gene. In certain embodiments, the mutant baculoviral genome may be produced by site-directed mutagenesis.

In certain embodiments, the edited gene can be generated by “knock-in” of a heterologous sequence that disrupts the reading frame of the WT baculovirus gene. For example, a selection marker expression cassette flanked by two flippase recognition targets (FRTs) can be PCR-amplified can be recombined into the capsid gene to disrupt the capsid via the lambda red system. After selection of the bacmid DNA and confirmation of the deletion, the selection marker expression cassette can be removed with the FLP-FRT recombination technology, leaving only one FRT site in the bacmid.

In some embodiments, the edited capsid sequence is generated with a gene-regulating system. Herein, the term “gene-regulating system” refers to a protein, nucleic acid, or combination thereof that is capable of modifying a target DNA sequence, thereby regulating the expression or function of the encoded gene product. Numerous gene-regulating systems suitable for use in the methods of the present invention are known in the art including, but not limited to, zinc-finger nuclease systems, TALEN systems, and CRISPR/Cas systems. As used herein, “regulate”, when used in reference to the effect of a gene-regulating system on a target gene, encompasses any change in the sequence of the endogenous target gene, and/or any change in the expression or function of the protein encoded by the endogenous target gene.

In some embodiments, the gene-regulating system may mediate a change in the sequence of the baculovirus gene, for example, by introducing one or more mutations into the gene, such as by insertion and/or deletion of one or more nucleic acids. Exemplary mechanisms that can mediate alterations of the capsid gene include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion.

In some embodiments, the gene-regulating system may mediate a change in the expression of a protein encoded by the baculovirus capsid gene. In such embodiments, the gene-regulating system may regulate the expression of the encoded capsid gene by modifications of the DNA sequence, or by acting on the mRNA product encoded by the DNA sequence. In some embodiments, the gene-regulating system may result in the expression of a modified baculovirus gene. In some embodiments, the expression level of the modified baculovirus gene may be decreased relative to the expression level of the unmodified baculovirus gene.

In some embodiments, the gene-regulating system is a nucleic acid-based gene-regulating system. Herein, a nucleic acid-based gene-regulating system is a system comprising one or more nucleic acid molecules that is capable of regulating the expression of the baculovirus gene without the requirement for an exogenous protein.

In some embodiments, the gene-regulating system is a protein-based gene-regulating system. Herein, a protein-based gene-regulating system is a system comprising one or more proteins capable of regulating the expression of the baculovirus gene in a sequence specific manner without the requirement for a nucleic acid guide molecule.

In some embodiments, the protein-based gene-regulating system comprises a protein comprising one or more zinc-finger binding domains and an enzymatic domain. Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416.

In some embodiments, the protein-based gene-regulating system comprises a protein comprising a Transcription activator-like effector nuclease (TALEN) domain and an enzymatic domain. Such embodiments are referred to herein as “TALENs.” TALEN-based systems comprise a protein comprising a TAL effector DNA binding domain and an enzymatic domain. They are made by fusing a TAL effector DNA-binding domain to a DNA cleavage domain (a nuclease which cuts DNA strands). The Fokl restriction enzyme described above is an exemplary enzymatic domain suitable for use in TALEN-based gene-regulating systems.

In some embodiments, CRISPR (Clustered Regularly Interspaced Short Palindromic Repeats)/Cas (CRISPR Associated) nuclease system may be used to edit the capsid gene. In some embodiments, a CRISPR-associated endonuclease (a “Cas” endonuclease) and a nucleic acid guide molecule (e.g., a guide RNA or gRNA) are employed. A Cas polypeptide refers to a polypeptide that can interact with a nucleic acid guide molecule and, in concert with the nucleic acid guide molecule, homes or localizes to a target DNA and includes naturally occurring Cas proteins and engineered, altered, or otherwise modified Cas proteins that differ by one or more amino acid residues from a naturally-occurring Cas sequence. In some embodiments, the Cas protein is a Cas9 protein. Cas9 is a multi-domain enzyme that uses an HNH nuclease domain to cleave the target strand of DNA and a RuvC-like domain to cleave the non-target strand. In some embodiments, mutants of Cas9 can be generated by selective domain inactivation enabling the conversion of WT Cas9 into an enzymatically inactive mutant (e.g., dCas9), which is unable to cleave DNA, or a nickase mutant, which is able to produce single-stranded DNA breaks by cleaving one or the other of the target or non-target strand. The precise location of the target modification site is determined by both (i) base-pairing complementarity between the gRNA and the target DNA sequence; and (ii) the location of a short motif, referred to as the protospacer adjacent motif (PAM), in the target DNA sequence. The PAM sequence is required for Cas binding to the target DNA sequence. A variety of PAM sequences are known in the art and are suitable for use with a particular Cas endonuclease (e.g., a Cas9 endonuclease) are known in the art (See e.g., Nat Methods. 2013 November; 10(11): 1116-1121 and Sci Rep. 2014; 4: 5405). In some embodiments, the Cas protein is a Cas9 protein or a Cas9 ortholog and is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the endonuclease is selected from the group consisting of C2C1, C2C3, Cpf1 (also referred to as Cas12a), CasI, CasIB, Cas2, Cas3, Cas4, Cas5, Cas6, Cas7, Cas8, Cas9 (also known as CsnI and Csx12), Cas10, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, CsxI7, CsxI4, Csx10, Csx16, CsaX, Csx3, CsxI, CsxI5, CsfI, Csf2, Csf3, and Csf4. Additional Cas9 orthologs are described in International PCT Publication No. WO 2015/071474.

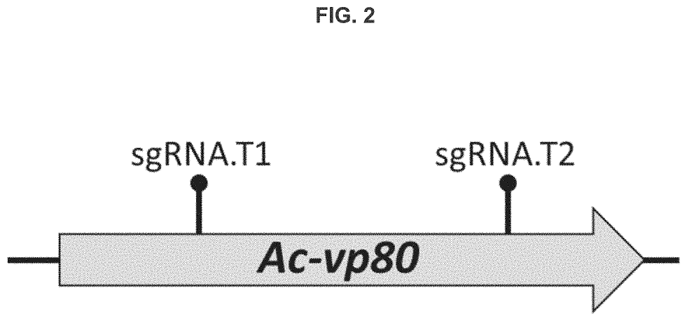

An exemplary genome-edited baculovirus DNA backbone according to the disclosure comprises an inactivated VP80 gene due to an insertion and/or deletion in the VP80 gene locus (see FIGS. 2-4). In certain embodiments, an inactivated VP80 gene due to an insertion and/or deletion in the VP80 gene locus is mediated by one or more gRNAs that target the VP80 gene locus. In certain embodiments, an inactivated VP80 gene due to an insertion and/or deletion in the VP80 gene locus is mediated by a gRNA comprising at least 75%, at least 80%, at least 85%, at least 90%, at least 95%, at least 99%, or 100% identity to the sequence CACGTTGACCAGCATGGTGT (SEQ ID NO:9). In certain embodiments, an inactivated VP80 gene due to an insertion and/or deletion in the VP80 gene locus is mediated by a gRNA comprising at least 75%, at least 80%, at least 85%, at least 90%, at least 95%, at least 99%, or 100% identity to the sequence GACGTGTCCAAGAAATTGAT (SEQ ID NO:10).

In certain exemplary embodiments, a genome-edited baculovirus DNA backbone according to the disclosure comprises an inactivated VP80 gene due to an insertion and/or deletion in the VP80 gene locus comprising the sequence set forth in SEQ ID NO:9 or SEQ ID NO:10. In certain exemplary embodiments, a genome-edited baculovirus DNA backbone according to the disclosure comprises an inactivated VP80 gene due to an insertion and/or deletion in a genomic sequence comprising the sequence set forth in SEQ ID NO:9 or SEQ ID NO:10. In certain exemplary embodiments, a genome-edited baculovirus DNA backbone according to the disclosure comprises an inactivated VP80 gene due to an insertion and/or deletion in a genomic sequence consisting of the sequence set forth in SEQ ID NO:9 or SEQ ID NO:10.

In certain embodiments, the recombinant bacmid of the disclosure further comprises at least one integration site (e.g., a mini-att Tn7 site) enabling the integration of an expression cassette (e.g., a ceDNA vector template) into the backbone. In certain embodiments, the recombinant bacmid is a “BIVVBac” as described in U.S. Patent Application No. 63/069,073 entitled “Baculovirus Expression System”, which is incorporated by reference herein. In certain embodiments that comprises at least two integration sites and allows the for the reduction in total number of baculovirus expression vectors that need to be generated. In certain embodiments, the bacmid further comprises a loxP site for integration of an additional gene by Cre-Lox mediated recombination.

In certain embodiments, the recombinant bacmid comprises a Rep gene (e.g., a B19 Rep gene). In certain embodiments, the Rep gene is expressed to facilitate the replication of a heterologous nucleic acid segment (e.g., a ceDNA vector) flanked by symmetric or assymetric AAV or non-AAV inverted terminal repeats (ITRs).

A recombinant bacmid may further comprise other elements required for its ability to be propagated in both bacterial (e.g., E. coli) and insect cells. In certain embodiment, the recombinant bacmid comprises a bacterial origin of replication or bacterial replicon. Various bacterial replicons are known to those of skill in the art, and include, for example, replicons of F plasmid derived origin. In certain embodiments, a suitable bacterial replicon is the mini-F replicon, which is a derivative of the F plasmid comprised of DNA regions oriS and incC required for replication and regulation. In certain embodiments, the bacterial replicon is a low-copy number replicon. In certain embodiments, the low-copy number replicon is a mini-F replicon.

Other elements of the recombinant bacmid of the disclosure includes one or more selectable marker sequences, and other reporter genes. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. In certain embodiments, the recombinant bacmid comprises a selectable marker sequence comprising an antibiotic resistance gene. In certain embodiments, the antibiotic resistance gene is a kanamycin resistance gene and confers resistance to kanamycin. Examples of reporters known and used in the art include: luciferase (Luc), green fluorescent protein (GFP), chloramphenicol acetyltransferase (CAT), β-galactosidase (LacZ), β-glucuronidase (Gus), and the like. In some cases, selectable markers can also be considered to be reporters. In certain embodiments, the recombinant bacmid comprises a reporter gene encoding a fluorescent protein. In certain embodiments, the fluorescent protein is a red fluorescent protein.

Those of skill in the art will recognize that the various elements of the recombinant bacmid described herein are in operable linkage with each other. Each of the various coding sequences in the bacmid may be in operable linkage with a regulatory region comprising, e.g., a promoter sequence. Any promoter sequence known in the art may be suitable. In certain embodiment, the promoter is a baculovirus-inducible promoter.

Genome Edited Recombinant Baculovirus Expression Vector (rBEV)

In certain embodiments, the disclosure provides a genome-edited recombinant baculovirus expression vector (rBEV) in which a foreign sequence (e.g., heterologous DNA sequence) is integrated in one or more of the integration sites of the genome edited bacmid describe supra. In certain embodiments, the foreign sequence is introduced into a Lox P site of the bacmid via Cre-Lox recombination. Inter other embodiments, the foreign sequence is inserted into the bacmid via Tn7-mediated transposition. Any foreign sequence (other than the functional counterpart of the attenuated or inactivated baculovirus gene) can be introduced into the genome edited bacmid in order to generate a genome-edited rBEV of the disclosure. In certain embodiments, the recombinant baculovirus expression vector comprises one or more of the ceDNA expression cassettes described below.

In certain embodiments, the recombinant baculovirus expression vector comprising the foreign sequence comprises a bacterial replicon, a first selectable marker sequence, a foreign sequence (e.g., a heterologous sequence) inserted into a first reporter gene, wherein the inserted foreign sequence disrupts the reading frame of the first reporter gene, a second reporter gene operably linked to a baculovirus-inducible promoter.

In certain embodiments, the rBEV comprises one AAV or non-AAV Rep genes (e.g., a B19 Rep). In certain embodiments, the rBEV comprises: a mini-F replicon; a first antibiotic resistance gene; a sequence encoding a Rep (e.g., a B19 Rep) inserted into a LacZa or functional portion thereof, wherein the inserted B19 Rep disrupts the reading frame of the LacZa or functional portion thereof; a gene encoding a fluorescent protein operably linked to a baculovirus-inducible promoter.

In certain embodiments, the rBEV comprises: a mini-F replicon; a first antibiotic resistance gene; a sequence encoding a GPV Rep inserted into a LacZa or functional portion thereof, wherein the inserted GPV Rep disrupts the reading frame of the LacZa or functional portion thereof; and a gene encoding a fluorescent protein operably linked to a baculovirus-inducible promoter. In certain embodiments, the recombinant bacmid comprises: a mini-F replicon; a first antibiotic resistance gene; a sequence encoding a AAV2 Rep inserted into a LacZa or functional portion thereof, wherein the inserted AAV2 Rep disrupts the reading frame of the LacZa or functional portion thereof; and a gene encoding a fluorescent protein operably linked to a baculovirus-inducible promoter.

In certain exemplary embodiments, for the purposes of producing a gene therapy vector, the rBEV comprises a transgene flanked by symmetric or assymetric AAV or non-AAV inverted terminal repeats (ITRs). In certain embodiments, the recombinant bacmid comprises: a mini-F replicon; an antibiotic resistance gene; a LacZa or functional portion thereof; a transgene flanked by symmetric or assymetric AAV or non-AAV ITRs.

In certain embodiments, for the purposes of producing a gene therapy vector, the rBEV comprises: a sequence encoding a B19 Rep inserted into a LacZa or functional portion thereof, wherein the inserted B19 Rep disrupts the reading frame of the LacZa or functional portion thereof; and a multiple cloning site comprising a heterologous sequence, wherein the heterologous sequence comprises from 5′ to 3′: a wild-type or truncated 5′ inverted terminal repeat derived from parvovirus B19; a sequence encoding a protein; one or more expression control sequences operably linked to the sequence encoding a protein; and a wild-type or truncated 3′ inverted terminal repeat derived from parvovirus B19.

In other embodiments, for the purposes of producing a gene therapy vector, the rBEV comprises: a sequence encoding a GPV Rep inserted into a LacZa or functional portion thereof, wherein the inserted GPV Rep disrupts the reading frame of the LacZa or functional portion thereof; and a multiple cloning site comprising a heterologous sequence, wherein the heterologous sequence comprises from 5′ to 3′: a wild-type or truncated 5′ inverted terminal repeat derived from GPV; a sequence encoding a protein; one or more expression control sequences operably linked to the sequence encoding a protein; and a wild-type or truncated 3′ inverted terminal repeat derived from GPV.

In other embodiments, for the purposes of producing a gene therapy vector, the rBEV comprises: a sequence encoding a AAV2 Rep inserted into a LacZa or functional portion thereof, wherein the inserted AAV2 Rep disrupts the reading frame of the LacZa or functional portion thereof; and a multiple cloning site comprising a heterologous sequence, wherein the heterologous sequence comprises from 5′ to 3′: a wild-type or truncated 5′ inverted terminal repeat derived from AAV2; a sequence encoding a protein; one or more expression control sequences operably linked to the sequence encoding a protein; and a wild-type or truncated 3′ inverted terminal repeat derived from AAV2.

### Providing a Functional Gene in Trans

In order to restore or “rescue” the replication of a modified bacmid or rBEV describe above, the disclosure provides a baculovirus expression system comprising the recombinant bacmid or baculovirus expression vector described above and functional protein (e.g., a functional capsid protein) which complements the inactivated or attenuated gene (e.g., inactivated capsid gene) of the recombinant bacmid or rBEV expression vector.

Accordingly, in certain embodiments, the functional gene (e.g., functional capsid gene) is provided in trans to the recombinant bacmid or baculovirus expression vector. In certain embodiments, the functional gene can be expressed by the host cell. Therefore, in certain embodiments, the disclosure provides host insect cells capable of rescuing the deficient baculovirus gene by expressing a complementing copy of the gene in trans. In certain embodiments, the disclosure provides an insect cell which expresses a complementing copy of the at least one gene essential for proper baculoviral virion assembly that is deficient in the baculovirus. For example, in certain embodiments, the disclosure provides a Sf9-derived cell line constitutively producing the product of the functional gene such that proper assembly of the baculovirus virion may be established. This recombinant cell line is used for production of baculovirus seed stock while conventional insect cell lines like Sf9, Sf21 or High-five cell lines can be infected with the produced baculovirus for heterologous expression of nucleic acid molecule (e.g., ceDNA vector). Accordingly, the baculovirus expression system of the invention also relates to an insect cell modified so as to express the functional counterpart of the baculovirus gene essential for proper baculovirus assembly, wherein the counterpart of the functional gene has been inactivated in the bacmid or baculovirus expression vector.

In a particular embodiment, the insect cell used for the production of the baculovirus is modified by transfection with an expression cassette coding for the functional counterpart of the gene essential for proper baculovirus virion assembly. In an embodiment, said expression cassette is integrated in the genome of said cell. One may also use insect cells transiently transfected with at least one plasmid comprising the expression cassette. Such an expression cassette may be a plasmid comprising the ORF of a gene essential for proper baculovirus virion assembly placed under the control of a promoter functional in the selected insect cell, and does not contain baculoviral genome sequences other than the gene essential for proper baculovirus virion assembly to be complemented and optionally the promoter sequence allowing the expression of said gene (in particular, an expression cassette is not a bacmid or any other baculoviral entire genome). Exemplary expression control sequences may be chosen among promoters, enhancers, insulators, etc. In one embodiment, the complementing gene is derived from the genome of the baculovirus in which the gene essential for proper baculovirus virion assembly has been made deficient. In another embodiment, the complementing gene originates from the genome of a different baculovirus species.

In yet another embodiment, the function counterpart of the gene essential for proper baculovirus virion assembly is placed under the control of an inducible promoter, allowing either the expression or repression of said gene under controlled conditions. In certain embodiments, the inducible promoter is a baculovirus-inducible promoter. In certain embodiments, the inducible promoter is the Autographa californica nucleopolyhedrovirus (AcMNPV) 39K promoter. In certain embodiments, the insect cell comprises an expression cassette which encodes a functional counterpart of a gene essential for baculovirus virion assembly that has been rendered deficient in the recombinant bacmid or rBEV.

ceDNA Expression Cassettes

In certain embodiments, the baculovirus expression vector system described herein can be used for the production of plasmid-like, capsid free, nucleic acid molecules useful for gene therapy. Therefore, in certain embodiments, the baculovirus expression vectors of the disclosure include the template DNA for a heterologous nucleic acid molecule that is a non-viral, capsid-free DNA vector with one or more covalently-closed ends (referred to herein as a “closed-ended DNA vectors” or “ceDNA vectors”, also known as a “closed-ended linear duplex DNA vector” or “CELiD DNA vectors”). The ceDNA vector may further comprise a transgene for delivery of a subject in need thereof.

In certain embodiments, a ceDNA vector is obtainable from a vector polynucleotide that encodes a heterologous nucleic acid operatively positioned between two inverted terminal repeat sequences (ITRs). In certain embodiments, the ceDNA vectors are formed from a continuous strand of complementary DNA with covalently-closed ends (linear, continuous and non-encapsidated structure), which comprise a 5′ inverted terminal repeat (ITR) sequence and a 3′ ITR sequence. In certain embodiments, the ITRs may be symmetrical with respect to each other. In other embodiments, the ITRs may be different, or asymmetrical with respect to each other. In certain embodiments, at least one of the ITRs comprises a terminal resolution site and a replication protein binding site (RPS) (sometimes referred to as a replicative protein binding site), e.g. a Rep binding site, and one of the ITRs comprises a deletion, insertion, or substitution with respect to the other ITR. In certain embodiments, at least one of the ITRs is an AAV ITR, e.g. a wild type AAV ITR or modified AAV ITR. In certain embodiments, at least one of the ITRs is a non-AAV ITR, e.g. a wild type non-AAV ITR or modified non-AAV ITR. In other embodiments, at least one of the ITRs is a modified ITR relative to the other ITR. In one embodiment, at least one of the ITRs is a non-functional ITR. In some embodiments, one of the ITRs is modified by deletion, insertion, and/or substitution as compared to a wild-type ITR sequence; and at least one of the ITRs comprises a functional terminal resolution site (trs) and a Rep binding site.

In certain embodiments, the disclosure is directed to a ceDNA vector template, comprising a first ITR, a second ITR, and a genetic expression cassette comprising a heterologous polynucleotide sequence, e.g., a transgene. In some embodiments, the first ITR and second ITR flank the genetic expression cassette. In some embodiment, the expression cassette comprises a cis-regulatory element, a promoter and at least one transgene. In some embodiments, the nucleic acid molecule does not comprise a gene encoding a capsid protein, a replication protein, and/or an assembly protein. In some embodiments, the genetic cassette encodes a therapeutic protein. In some embodiments, the therapeutic protein comprises a clotting factor. In some embodiments, the genetic cassette encodes a miRNA. In certain embodiments, the genetic cassette is positioned between the first ITR and the second ITR. In some embodiments, the nucleic acid molecule further comprises one or more noncoding regions. In certain embodiments, the one or more non-coding region comprises a promoter sequence, an intron, a post-transcriptional regulatory element, a 3′UTR poly(A) sequence, or any combination thereof. In one embodiment, the expression cassette is a single stranded nucleic acid. In another embodiment, the genetic cassette is a double stranded nucleic acid.

In certain embodiments, the template encoding a ceDNA vector comprises, in the 5′ to 3′ direction: a first inverted terminal repeat (ITR), a nucleotide sequence of interest (for example an expression cassette or transgene as described herein) and a second ITR, wherein the first ITR and the second ITR are asymmetric with respect to each other—that is, they are different from one another. As an exemplary embodiment, the first ITR can be a wild-type ITR and the second ITR can be a mutated or modified ITR. In some embodiments, the first ITR can be a mutated or modified ITR and the second ITR a wild-type ITR. In another embodiment, the first ITR and the second ITR are both modified but are different sequences, or have different modifications, or are not identical modified ITRs.

In some embodiments, a ceDNA vector described herein comprising the expression cassette with a transgene, which can be, for example, a regulatory sequence, a sequence encoding a nucleic acid (e.g., such as a miR or an antisense sequence), or a sequence encoding a polypeptide (e.g., such as a transgene). In one embodiment, the transgene encodes a theraputic protein, wherein the therapeutic protein comprises a Factor VIII (FVIII) polypeptide. In one embodiment, the transgene may be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other.

In one embodiment in each of these aspects, an expression cassette is located between two ITRs comprised in the following order with one or more of: a promoter operably linked to a transgene, a posttranscriptional regulatory element, and a polyadenylation and termination signal. In one embodiment, the promoter is regulatable, inducible or repressible. The posttranscriptional regulatory element is a sequence that modulates expression of the transgene, as a non-limiting example, any sequence that creates a tertiary structure that enhances expression of the transgene.

The ceDNA expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene or nucleic acid in the range of 500 to 50,000 nucleotides in length. In some embodiments, the expression cassette can comprise a transgene or nucleic acid in the range of 500 to 75,000 nucleotides in length. In some embodiments, the expression cassette can comprise a transgene or nucleic acid is in the range of 500 to 10,000 nucleotides in length. In some embodiments, the expression cassette can comprise a transgene or nucleic acid is in the range of 1000 to 10,000 nucleotides in length. In some embodiments, the expression cassette can comprise a transgene or nucleic acid is in the range of 500 to 5,000 nucleotides in length.

In some embodiments, the nucleic acid molecule comprises an expression cassette encoding a therapeutic protein. In some embodiments, the therapeutic protein comprises a clotting factor. In some embodiments, the expression cassette encodes a miRNA. In some embodiments, the expression cassette comprises at least one noncoding region. In certain embodiments, non-coding region comprises a promoter sequence, an intron, a post-transcriptional regulatory element, a 3′UTR poly(A) sequence, or any combination thereof.

In some embodiments, the genetic cassette comprises a nucleotide sequence encoding a codon optimized FVIII driven by a mTTR promoter. In some embodiments, the mTTR promoter comprises the nucleic acid sequence of SEQ ID NO: 17. In some embodiments, the genetic cassette further comprises an A1MB2 enhancer element. In some embodiments, the A1MB2 enhancer element comprises the nucleic acid sequence of SEQ ID NO: 16. In some embodiments, the genetic cassette further comprises a chimeric or synthetic intron. In some embodiments, the chimeric intron consists of chicken beta-actin/rabbit beta-globin intron and has been modified to eliminate five existing ATG sequences to reduce false translation starts. In some embodiments, the intronic sequence is positioned 5′ to the nucleic acid sequence encoding the FVIII polypeptide. In some embodiments, the chimeric intron is positioned 5′ to a promoter sequence, such as the mTTR promoter. In some embodiments, the chimeric intron comprises the nucleic acid sequence of SEQ ID NO: 18. In some embodiments, the genetic cassette further comprises a a Woodchuck Posttranscriptional Regulatory Element (WPRE). In some embodiments, the WPRE comprises the nucleic acid sequence of SEQ ID NO: 19. In some embodiments, the genetic cassette further comprises a Bovine Growth Hormone Polyadenylation (bGHpA) signal. In some embodiments, the bGHpA signal comprises the nucleic acid sequence of SEQ ID NO: 20. In some embodiments, the genetic cassette comprises a nucleotide sequence having at least 70%, 75%, 80%, 85%, 90%, 95%, or 100% sequence identity to SEQ ID NO: 14. In some embodiments, the genetic cassette comprises the nucleotide sequence of SEQ ID NO: 14.

### Inverted Terminal Repeats

As disclosed herein, ceDNA vectors contain a heterologous gene positioned between two inverted terminal repeat (ITR) sequences. In certain embodiments, the 5′ ITR and the 3′ ITR are adeno-associated virus (AAV) ITRs or non-AAV ITRs. In certain embodiments, non-AAV ITRs are ITRs obtained from a member of the viral family Parvoviridae. Suitable ITR sequences include AAV ITRs of AAV serotypes known to those of skill in the art. Exemplary AAV and non-AAV ITR sequences for use in the ceDNA vectors are disclosed in WO2019/051255, WO2019032898A1, WO2020033863A1, and WO2017152149A1, and U.S. Patent Application No. 63/069,114, the disclosures of which are herein incorporated by reference in their entireties.

The ceDNA vectors of the disclosure may employ ITR sequences from any known parvovirus, for example a dependovirus such as AAV (e.g., AAV1, AAV2, AAV3, AAV4, AAV5, AAV 6, AAV7, AAV8, AAV9, AAV10, AAV11, AAV12, AAVrh8, AAVrhIO, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvoviris (GenBank Accession No: NC 000883), Minute Virus from Mouse (MVM) (GenBank Accession No. NC 001510); goose parvovirus (GenBankAccession No. NC 001701); snake parvovirus 1 (GenBank Accession No. NC 006148).

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

In certain embodiments, ITRs are obtained from a member of the viral family Parvoviridae. For example, non-AAV ITR sequences may be derived from Goose parvovirus (GPV) or parvovirus B19. In some embodiments, the ITR is not derived from an AAV genome. In some embodiments, the ITR is an ITR of a non-AAV. In some embodiments, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain embodiments, the ITR is derived from erythrovirus parvovirus B19 (human virus). In another embodiment, the ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY. In some embodiments, the ITR is derived from a porcine parvovirus, e.g., porcine parvovirus U44978. In some embodiments, the ITR is derived from a mice minute virus, e.g., mice minute virus U34256. In some embodiments, the ITR is derived from a canine parvovirus, e.g., canine parvovirus M19296. In some embodiments, the ITR is derived from a mink enteritis virus, e.g., mink enteritis virus 000765. In some embodiments, the ITR is derived from a Dependoparvovirus. In one embodiment, the Dependoparvovirus is a Dependovirus Goose parvovirus (GPV) strain. In a specific embodiment, the GPV strain is attenuated, e.g., GPV strain 82-0321V. In another specific embodiment, the GPV strain is pathogenic, e.g., GPV strain B. Examples of suitable Parvoviral ITR sequences are set forth in Table 1.

TABLE 1
Parvoviral ITR Sequences

| SEQ | | | |
|---|---|---|---|
| ID | | | |
| NO: | Parvovirus | Descriptor | Sequence |
| 1 | B19 | B19Δ135 | CTCTGGGCCAGCTTGCTTGGGGTTGCCTTGACACTAAGACA |
| | | | AGCGGCGCGCCGCTTGATCTTAGTGGCACGTCAACCCCAA |
| | | | GCGCTGGCCCAGAGCCAACCCTAATTCCGGAAGTCCCGCC |
| | | | CACCGGAAGTGACGTCACAGGAAATGACGTCACAGGAAAT |
| | | | GACGTAATTGTCCGCCATCTTGTACCGGAAGTCCCGCCTAC |
| | | | CGGCGGCGACCGGCGGCATCTGATTTGGTGTCTTCTTTTAA |
| | | | ATTTT |
| 2 | | B19.WT | CCAAATCAGATGCCGCCGGTCGCCGCCGGTAGGCGGGAC |
| | | | TTCCGGTACAAGATGGCGGACAATTACGTCATTTCCTGTGA |
| | | | CGTCATTTCCTGTGACGTCACTTCCGGTGGGCGGGACTTCC |
| | | | GGAATTAGGGTTGGCTCTGGGCCAGCTTGCTTGGGGTTGC |
| | | | CTTGACACTAAGACAAGCGGCGCGCCGCTTGATCTTAGTG |
| | | | GCACGTCAACCCCAAGCGCTGGCCCAGAGCCAACCCTAAT |
| | | | TCCGGAAGTCCCGCCCACCGGAAGTGACGTCACAGGAAAT |
| | | | GACGTCACAGGAAATGACGTAATTGTCCGCCATCTTGTACC |
| | | | GGAAGTCCCGCCTACCGGCGGCGACCGGCGGCATCTGATT |
| | | | TGGTGTCTTCTTTTAAATTTT |
| 3 | GPV | GPVΔ162 | CGGTGACGTGTTTCCGGCTGTTAGGTTGACCACGCGCATG |
| | | | CCGCGCGGTCAGCCCAATAGTTAAGCCGGAAACACGTCAC |
| | | | CGGAAGTCACATGACCGGAAGTCACGTGACCGGAAACACG |
| | | | TGACAGGAAGCACGTGACCGGAACTACGTCACCGGATGTG |
| | | | CGTCACCGGAAGCATGTGACCGGAACTTGCGTCACTTCCC |
| | | | CCTCCCCTGATTGGCTGGTTCGAACGAACGAACCCTCCAAT |
| | | | GAGACTCAAGGACAAGAGGATATTTTGCGCGCCAGGAAGT |
| | | | G |
| 4 | | GPV.WT | CTCATTGGAGGGTTCGTTCGTTCGAACCAGCCAATCAGGG |
| | | | GAGGGGGAAGTGACGCAAGTTCCGGTCACATGCTTCCGGT |
| | | | GACGCACATCCGGTGACGTAGTTCCGGTCACGTGCTTCCT |
| | | | GTCACGTGTTTCCGGTCACGTGACTTCCGGTCATGTGACTT |
| | | | CCGGTGACGTGTTTCCGGCTGTTAGGTTGACCACGCGCAT |
| | | | GCCGCGCGGTCAGCCCAATAGTTAAGCCGGAAACACGTCA |
| | | | CCGGAAGTCACATGACCGGAAGTCACGTGACCGGAAACAC |
| | | | GTGACAGGAAGCACGTGACCGGAACTACGTCACCGGATGT |
| | | | GCGTCACCGGAAGCATGTGACCGGAACTTGCGTCACTTCC |
| | | | CCCTCCCCTGATTGGCTGGTTCGAACGAACGAACCCTCCAA |
| | | | TGAGACTCAAGGACAAGAGGATATTTTGCGCGCCAGGAAG |
| | | | TG |
| 5 | AAV2 | AAV2.WT (5′) | AGGAACCCCTAGTGATGGAGTTGGCCACTCCCTCTCTGCG |
| | | | CGCTCGCTCGCTCACTGAGGCCGCCCGGGCAAAGCCCGG |
| | | | GCGTCGGGCGACCTTTGGTCGCCCGGCCTCAGTGAGCGA |
| | | | GCGAGCGCGCAGAGAGGGAGTGGCCAA |
| 6 | | AAV2.WT (3′) | AGGAACCCCTAGTGATGGAGTTGGCCACTCCCTCTCTGCG |
| | | | CGCTCGCTCGCTCACTGAGGCCGGGCGACCAAAGGTCGC |
| | | | CCGACGCCCGGGCTTTGCCCGGGCGGCCTCAGTGAGCGA |
| | | | GCGAGCGCGCAGAGAGGGAGTGGCCAA |
| 7 | | AAV2Δ15 | AGGAACCCCTAGTGATGGAGTTGGCCACTCCCTCTCTGCG |
| | | | CGCTCGCTCGCTCACTGAGGCCGGGCGACCAAAGGTCGC |
| | | | CCGACGCCCGGGCTTTGCCCGGGCGGCCTCAGTGAGCGA |
| | | | GCGAGCGCGCAG |
| 8 | | AAV2Δ15Δ11 | AGGAACCCCTAGTGATGGAGTTGGCCACTCCCTCTCTGCG |
| | | | CGCTCGCTCGCTCACTGAGGCCGGGCGACCAAAGGTCGC |
| | | | CCGACGCCCGGGCGGCCTCAGTGAGCGAGCGAGCGCGCA |
| | | | G |
| 12 | HBoV1 | HBoV1 5′ ITR | GTGGTTGTACAGACGCCATCTTGGAATCCAATATGTCTGCC |
| | | | GGCTCAGTCATGCCTGCGCTGCGCGCAGCGCGCTGCGCG |
| | | | CGCGCATGATCTAATCGCCGGCAGACATATTGGATTCCAAG |
| | | | ATGGCGTCTGTACAACCAC |
| 13 | HBoV1 | HBoV1 3′ ITR | TTGCTTATGCAATCGCGAAACTCTATATCTTTTAATGTGTTG |
| | | | TTGTTGTACATGCGCCATCTTAGTTTTATATCAGCTGGCGCC |
| | | | TTAGTTATATAACATGCATGTTATATAACTAAGGCGCCAGCT |
| | | | GATATAAAACTAAGATGGCGCATGTACAACAACAACACATTA |
| | | | AAAGATATAGAGTTTCGCGATTGCATAAGCAA |

The wild-type or mutated or otherwise modified ITR sequences provided herein represent DNA sequences included in the baculovirus expression construct (e.g., the ceDNA template DNA), for production of the ceDNA vector. Thus, ITR sequences actually contained in the ceDNA vector produced by the baculovirus expression construct may or may not be identical to the ITR sequences provided herein as a result of naturally occurring changes taking place during the production process (e.g., replication error).

### Transgenes

In certain embodiments, the nucleic molecules (e.g., ceDNA vectors) can deliver and encode one or more transgenes in a target cell. The transgenes can be protein encoding transcripts, non-coding transcripts, or both. The nucleic acid molecules can comprise multiple coding sequences, and a non-canonical translation initiation site or more than one promoter to express protein encoding transcripts, non-coding transcripts, or both. The transgene can comprise a sequence encoding more than one protein, or can be a sequence of a non-coding transcript.

The expression cassette can comprise any transgene of interest. Transgenes of interest include but are not limited to, nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the transgenes in the expression cassette encodes one or more polypeptides, peptides, ribozymes, peptide nucleic acids, siRNAs, RNAis, antisense oligonucleotides, antisense polynucleotides, antibodies, antigen binding fragments, or any combination thereof. In some embodiments, the transgene is a therapeutic gene, or a marker protein. In some embodiments, the transgene is an agonist or antagonist. In some embodiments, the antagonist is a mimetic or antibody, or antibody fragment, or antigen-binding fragment thereof, e.g., a neutralizing antibody or antibody fragment and the like. In some embodiments, the transgene encodes an antibody, including a full-length antibody or antibody fragment, as defined herein. In some embodiments, the antibody is an antigen-binding domain or an immunoglobulin variable domain sequence.

In some embodiments, a transgene described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using publicly available databases.

In certain embodiments the expression construct encodes a transgene encodes a therapeutic protein. In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

Any therapeutic protein can be produced by a baculovirus expression vector system of the present disclosure, including, without limitation, production of clotting factor. In some embodiments, the clotting factor is selected from the group consisting of FI, FII, FIII, FIV, FV, FVI, FVII, FVIII, FIX, FX, FXI, FXII, FXIII, VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI-2), any zymogen thereof, any active form thereof, and any combination thereof. In one embodiment, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

### Growth Factors

In some aspects, provided herein is the production of a nucleic acid molecule comprising a first ITR, a second ITR, and a genetic cassette encoding a target sequence, wherein the target sequence encodes a therapeutic protein, and wherein the therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7. In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin B1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is fetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP)). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

Micro RNAs (miRNAs)

MicroRNAs (miRNAs) are small non-coding RNA molecules (about 18-22 nucleotides) that negatively regulate gene expression by inhibiting translation or inducing messenger RNA (mRNA) degradation. Since their discovery, miRNAs have been implicated in various cellular processes including apoptosis, differentiation and cell proliferation and they have shown to play a key role in carcinogenesis. The ability of miRNAs to regulate gene expression makes expression of miRNAs in vivo a valuable tool in gene therapy.

In some aspects, provided herein is the production of a nucleic acid molecule comprising a first ITR, a second ITR, and a genetic cassette encoding a target sequence, wherein the target sequence encodes a miRNA, and wherein the first ITR and/or the second ITR are an ITR of a non-adeno-associated virus (e.g., the first ITR and/or the second ITR are from a non-AAV). The miRNA can be any miRNA known in the art. In some embodiments, the miRNA down regulates the expression of a target gene. In certain embodiments, the target gene is selected from SOD1, HTT, RHO, or any combination thereof.

In some embodiments, the genetic cassette encodes one miRNA. In some embodiments, the genetic cassette encodes more than one miRNA. In some embodiments, the genetic cassette encodes two or more different miRNAs. In some embodiments, the genetic cassette encodes two or more copies of the same miRNA. In some embodiments, the genetic cassette encodes two or more variants of the same therapeutic protein. In certain embodiments, the genetic cassette encodes one or more miRNA and one or more therapeutic protein.

In some embodiments, the miRNA is a naturally occurring miRNA. In some embodiments, the miRNA is an engineered miRNA. In some embodiments, the miRNA is an artificial miRNA. In certain embodiments, the miRNA comprises the miHTT engineered miRNA disclosed by Evers et al., Molecular Therapy 26(9):1-15 (epub ahead of print June 2018). In certain embodiments, the miRNA comprises the miR SOD1 artificial miRNA disclosed by Dirren et al., Annals of Clinical and Translational Neurology 2(2):167-84 (February 2015). In certain embodiments, the miRNA comprises miR-708, which targets RHO (see Behrman et al., JCB 192(6):919-27 (2011).

In some embodiments, the miRNA upregulates expression of a gene by down regulating the expression of an inhibitor of the gene. In some embodiments, the inhibitor is a natural, e.g., wild-type, inhibitor. In some embodiments, the inhibitor results from a mutated, heterologous, and/or misexpressed gene.

### Expression Control Elements

In some embodiments, the nucleic acid molecule or vector produced by a baculovirus expression vector system described herein further comprises at least one expression control sequence. An expression control sequence as used herein is any regulatory nucleotide sequence, such as a promoter sequence or promoter-enhancer combination, which facilitates the efficient transcription and translation of the coding nucleic acid to which it is operably linked. For example, the isolated nucleic acid molecule produced by a method of the disclosure can be operably linked to at least one transcription control sequence.

The gene expression control sequence can, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus.

Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. Examples of liver specific promoters include, but are not limited to, a mouse transthyretin promoter (mTTR), a native human factor VIII promoter, a native human factor IX promoter, human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The FVIII promoter is described in Figueiredo and Brownlee, 1995, J. Biol. Chem. 270:11828-11838. In certain embodiments, the promoter comprises any of the mTTR promoters (e.g., mTTR202 promoter, mTTR202opt promoter, mTTR482 promoter) as disclosed in U.S. patent publication no. US2019/0048362, which is incorporated by reference herein in its entirety.

In some embodiments, the nucleic acid molecule comprises a tissue specific promoter. In certain embodiments, the tissue specific promoter drives expression of the therapeutic protein, e.g., the clotting factor, in the liver, e.g., in hepatocytes and/or endothelial cells. In particular embodiments, the promoter is selected from the group consisting of a mouse transthyretin promoter (mTTR), a native human factor VIII promoter, a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, a phosphoglycerate kinase (PGK) promoter and any combination thereof. In some embodiments, the promoter is selected from a liver specific promoter (e.g., α1-antitrypsin (AAT)), a muscle specific promoter (e.g., muscle creatine kinase (MCK), myosin heavy chain alpha (αMHC), myoglobin (MB), and desmin (DES)), a synthetic promoter (e.g., SPc5-12, 2R5Sc5-12, dMCK, and tMCK) and any combination thereof.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In a particular example, a promoter useful for the disclosure is an ET promoter, which is also known as GenBank No. AY661265. See also Vigna et al., Molecular Therapy 11(5):763 (2005). Examples of other suitable vectors and expression control sequences are described in WO 02/092134, EP1395293, or U.S. Pat. Nos. 6,808,905, 7,745,179, or 7,179,903, which are incorporated by reference herein in their entireties.

In general, the expression control sequences shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operablyjoined coding nucleic acid. The gene expression sequences optionally include enhancer sequences or upstream activator sequences as desired.

Additional cis-regulatory elements include, but are not limited to, a riboswitch, an insulator, a mir-regulatable element, a post-transcriptional regulatory element (e.g., WPRE), and a polyadenylation and termination signal (e.g., BGH poly A). In certain embodiments, the expression cassette can also comprise an internal ribosome entry site (IRES) and/or a 2A element. In some embodiments, the ceDNA vector comprises additional components to regulate expression of the transgene, for example, a regulatory switch which is a kill switch to enable controlled cell death of a cell comprising a ceDNA vector.

In certain embodiments, a nucleic acid molecule produced by a baculovirus expression vector system described herein comprises one or more miRNA target sequences which, for example, are operably linked to a transgene.

In some embodiments, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In some embodiments, the target sequence is an miR-142 target. In some embodiments, the complementary sequence of hematopoietic-specific microRNAs, such as miR-142 (142T), is incorporated into a nucleic acid molecule comprising a transgene, making the transgene-encoding transcript susceptible to miRNA-mediated down-regulation. By this method, transgene expression can be prevented in hematopoietic-lineage antigen presenting cells (APC), while being maintained in non-hematopoietic cells. This strategy can impose a stringent post-transcriptional control on transgene expression and thus enables stable delivery and long-term expression oftransgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### Host Cells

The baculovirus expression system of the disclosure can be propagated to produce non-viral capsid free ceDNA molecules can be produced in permissive host cells.

Suitable host cells are known to those of skill in the art. A “host cell” refers to any cell that harbors, or is capable of harboring, any substance of interest.

In some embodiments, host cells suitable for use are of insect origin. In some embodiments, a suitable insect host cell includes, for example, a cell line isolated from Spodoptera frugiperda or a cell line isolated from Trichoplusia ni. Exemplary insect host cells include, without limitation, Sf9 cells, Sf21 cells, Express Sf+ cells, and S2 cells from the Fall Army worm (Spodoptera frugiperda), or BTI-TN-5B1-4 (High Five cells) from the cabbage looper Trichoplusia ni (Lepidoptera), D. melanogaster, and other cell lines. In one particular embodiment, the insect host cells are Sf9 cells. These cells are commercially available from a number of sources (e.g., ThermoFisher Scientific, ATCC, and Expression Systems). Other suitable host insect cells are known to those of skill in the art.

rBV infects insect cells upon contact under conditions conducive from the virus to enter the cell, e.g., by culturing the contacted cells at about 28° C. for about three days in a medium conducive for expression of the foreign proteins, e.g., in Gibco insect media (ExpiSf CD Medium, Sf-900 III SFM, Express Five SFM, or SF-900 II SEM (ThermoFisher Scientific), ESF921 or ESF AF media (Expression Systems). Successful infection can be monitored e.g., by expression of a visually detectable selection marker protein, or the expression of the gene for which had been incorporated into the rBV genome.

Host cells comprising vectors of the disclosure are grown in an appropriate growth medium. As used herein, the term “appropriate growth medium” means a medium containing nutrient required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). Insect cells may be cultured in a medium conducive for maintenance and growth, such as, but not limited to Gibco insect media ExpiSf CD Medium, Sf-900 III SFM, Express Five SFM, or SF-900 II SEM (ThermoFisher Scientific), ESF921 and ESFAF (Expression Systems). The growth medium will generally select for cells containing the vector by, for example, drug selection or deficiency in an essential nutrient which is complemented by the selectable marker on the vector.

ceDNA Vector Expression and Isolation

In certain aspects, the disclosure relates to production of nucleic acid molecules (e.g., ceDNA vectors) described herein by propagating the baculovirus expression vectors described here. In certain embodiments, the capsid free non-viral DNA vector (ceDNA vector) is obtained by propagating a baculovirus expression vector comprising a polynucleotide expression construct template comprising in this order: a first 5′ ITR; an expression cassette; and a 3′ ITR. In one embodiment, at least one of the 5′ and 3′ ITR is a modified ITR, or where when both the 5′ and 3′ ITRs are modified, they have different modifications from one another and are not the same sequence, i.e., they are asymmetric. In certain embodiments, the ITR sequences are from a virus selected from a parvovirus, a dependovirus, and an adeno-associated virus (AAV). In certain embodiments, ITRs are from different viral serotypes.

In certain embodiments, the baculovirus expression vectors are propagated in a permissive host cell (e.g., an insect cell), in the presence of a Rep protein. In certain embodiments, the polynucleotide template replicates in the host cell to produce ceDNA vectors. ceDNA vector production undergoes two steps: first, excision (“rescue”) of template from the template backbone via Rep proteins, and second, Rep mediated replication of the excised ceDNA vector. Rep proteins and Rep binding sites for particular ITR sequences are well known to those of ordinary skill in the art. One of ordinary skill understands to choose a Rep protein from a serotype that binds to and replicates the nucleic acid sequence based upon at least one functional ITR. For example, if the replication competent ITR is from AAV serotype 2, the corresponding Rep would be from an AAV serotype that works with that serotype such as AAV2 ITR with AAV2 or AAV4 Rep but not AAV5 Rep, which does not. Upon replication, the covalently-closed ended DNA vector continues to accumulate in permissive cells and ceDNA vector is preferably sufficiently stable overtime in the presence of Rep protein under standard replication conditions, e.g. to accumulate in an amount that is at least 1 pg/cell, preferably at least 2 pg/cell, preferably at least 3 pg/cell, more preferably at least 4 pg/cell, even more preferably at least 5 pg/cell.

Accordingly, in one aspect, the production process comprising the steps of: a) incubating a population of host cells (e.g. insect cells) with a baculovirus expression vector described herein, in the presence of a Rep protein under conditions effective and for a time sufficient to induce production of the ceDNA vector within the host cells, and b) harvesting and isolating the ceDNA vector from the host cells. The presence of Rep protein induces replication of the vector polynucleotide with a modified ITR to produce the ceDNA vector in a host cell.

In certain embodiments, Rep is added to host cells at a MOI of about 3. In certain embodiments, baculovirus expression vector is used to deliver both the polynucleotide that encodes Rep protein and the non-viral DNA vector polynucleotide expression construct template for ceDNA. In other embodiments, the host cell is engineered to express Rep protein.

ceDNA vectors can be obtained from infected insect cells by lysing the cells and harvesting the ceDNA vectors. Lysing can be accomplished with physical force (e.g., with a French Press or sonication), detergent-containing lysis buffer, or enzymatic digestion of the cell matrix with, e.g., chitinase that is naturally expressed by the baculovirus genome. The ceDNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

### Methods of Use

A baculovirus expression vector system provided herein finds use in the production of a product encoded by a foreign sequence inserted in a recombinant bacmid described herein. Scalable production of the product can be achieved by several approaches known in the art.

One approach comprises the infection of suitable insect host cells that supports the growth of baculovirus. In certain embodiments, a recombinant bacmid comprising foreign sequences are described herein is first propagated in a suitable bacterial host cell (e.g., E. coli). The recombinant bacmid is then isolated from the bacterial host cell and transfected into a suitable insect host cell using a suitable transfection reagent (e.g., CELLFECTIN). The insect host cell generates recombinant baculovirus particles which can then be infected into a host insect cell for viral amplification of the foreign sequences.

In certain embodiments, provided herein is a method of producing a product encoded by a foreign sequence, comprising transfecting a recombinant bacmid described herein into a suitable insect cell under appropriate conditions to generate a recombinant baculovirus; and infecting a second suitable insect cell with the recombinant baculovirus under appropriate conditions to produce the product encoded by the foreign sequence. In certain embodiments, for the purposes of producing a gene therapeutic, the recombinant bacmid comprises a Rep coding sequence and a sequence encoding a protein flanked on both sides by ITRs.

In certain embodiments, provided herein is a method of producing a nucleic acid molecule, comprising transfecting a recombinant bacmid described herein into a suitable insect cell under appropriate conditions to generate a recombinant baculovirus; and infecting a second suitable insect cell with the recombinant baculovirus under appropriate conditions to produce the nucleic acid molecule. In certain embodiments, provided herein is a method of producing a ceDNA, comprising transfecting a recombinant bacmid described herein into a suitable insect cell under appropriate conditions to generate a recombinant baculovirus; and infecting a second suitable insect cell with the recombinant baculovirus under appropriate conditions to produce the ceDNA.

In another approach, a stable cell line can be generated by stably integrating a protein encoding sequence under the control of a baculovirus gene promoter (e.g., a baculovirus constitutive gene promoter). In certain embodiments, the stable cell line is a stable insect cell line. Stable integration of sequences can be performed by any method known to those of skill in the art. Methods for stable integration of nucleic acids into a variety of host cell lines are known in the art (see Examples below for more detailed description of an exemplary producer cell line created by stable integration of nucleic acids). For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A. C. et al. (2000) Proc. Natl. Acad. Sci. 97:5995-6000).

In the stable cell line approach, in one embodiment, a BEV encoding a complement protein required for proper expression of the protein encoding sequence is introduced into the stable cell line. In certain embodiments, for the purposes of producing a gene therapeutic, the stable cell line comprises a therapeutic protein-coding gene with flanking symmetric or assymetric AAV or non-AAV ITRs stably integrated therein. A BEV comprising encoding a suitable Rep is then introduced into the stable cell line under conditions necessary for the production of the gene therapeutic.

Exemplary methods of generating specific stable cell lines are described in U.S. Patent Application No. 63/069,073.

In yet another approach, production of product encoded by a foreign sequence can be achieved using a stable cell line in a baculovirus-free manner. In certain embodiments, for the purposes of producing a gene therapeutic, the stable cell line comprises a therapeutic protein-coding gene with flanking symmetric or assymetric AAV or non-AAV ITRs stably integrated therein. In certain embodiments, baculovirus-free production in the stable cell lines comprises transient expression of a Rep protein in the stable cell line under the control of a baculovirus gene promoter. Suitable baculovirus gene promoters are known to those of skill in the art. In certain embodiments, the baculovirus gene promoter is an immediate-early (ie) gene promoter of Orgyia pseudotsugata multiple nucleopolyhedrovirus (OpMNPV). In certain embodiments, the baculovirus gene promoter is the OplE2 promoter of OpMNPV. Various methods of mediating transient gene expression are known to those of skill in the art. In certain embodiments, transient gene expression can be achieved by polyethylenimine (PEI)-mediated trasfection.

Downstream purification of the product can involve any methods known to those of skill in the art. For example, for viral or non-viral vectors for gene therapy purposes may be purified by plasmid DNA isolation kits containing silica-based column that separate the low molecular weight DNA from the RNAs, high molecular weight DNAs, proteins, and other impurities by ion-exchange chromatography.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

### EXAMPLES

Having provided the foregoing disclosure, a further understanding can be obtained by reference to the examples provided herein. These examples are for purposes of illustration only and are not intended to be limiting.

### Example 1: Closed Ended DNA (ceDNA) Vector Yield Improvement

In the baculovirus-insect cell system, recombinant BEV delivers the gene of interest under a strong promoter and provides transcriptional complex essential for the virus replication in insect cells. Typically, the baculovirus DNA genome replicates in the nucleus and produces several tens of millions of progeny virus particles, each containing a full-length DNA genome. It has been demonstrated that baculoviral genomic DNAs are co-purified with the ceDNA while isolating DNA from the insect cells using a plasmid DNA-based purification method such as silica gel columns. The commercial plasmid DNA kit columns are generally not designed to separate DNA based on their molecular weights and therefore, typically, all forms of DNA present in the sample can bind to these columns. Moreover, the binding capacity of large molecular weight DNA could be different than the low molecular weight DNA and the anion-exchange based kit columns are not optimized for the binding efficiency of different size DNAs.

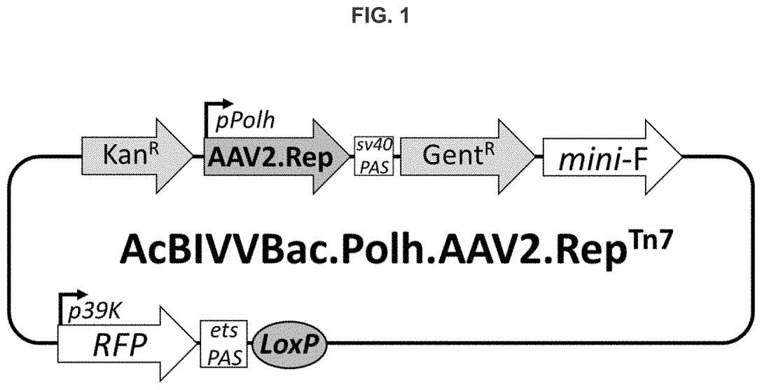

It was hypothesized that the high molecular weight DNA (>20 kb) observed in ceDNA preps were most likely baculoviral genomic DNA that were co-purified with the low molecular weight ceDNA (˜7 kb). To test the hypothesis, an indirect approach of knocking out an essential gene of the baculovirus genome that is required for producing infectious virus particles in insect cells (Sf9) was used. This approach would reduce the number of progeny virus particles produced and ultimately, the baculoviral genomic DNA contamination in the ceDNA preparations. The AcMNPV vp80 capsid gene was targeted, which is essential for progeny (budded virus) virus production and infection in Sf9 cells. As a proof-of-concept, a AcBIVVBac.Polh.AAV2.RepTn7 BEV (FIG. 1) (see U.S. Ser. No. 63/069,073 entitled “Baculovirus Expression System”, which is incorporated by reference herein), was used to knock out vp80 gene by the CRISPR-Cas9 system. Subsequently, a complement Sf9 cell line expressing VP80 under the AcMNPV-inducible 39K promoter to produce working BEV stock (P2) of the vp80 knock out (KO) virus was also made. This would allow for the vp80KO AcBIVVBac.Polh.AAV2.RepTn7 BEV to undergo one round of replication and could initiate the AAV ITR-mediated ceDNA vector genome replication in Sf9 cells.

### Example 2: CRISPR-Cas Knock-Out of the AcMNPV VP80

To knock-out the AcMNPV vp80 gene, two crRNAs targeting the coding sequence were designed and used for generating functional sgRNAs using the Alt-R CRISPR-Cas9 system (Integrated DNA Technology™), according to the manufacturer's instructions (FIG. 2 and Table 2).

TABLE 2
sgRNA Sequences Targeting VP80

| SEQ ID NO: | Descriptor | Sequence (5′-3′) |
|---|---|---|
| 9 | crRNA_VP80t1 | CACGTTGACCAGCATGGTGT |
| 10 | crRNA_VP80t2 | GACGTGTCCAAGAAATTGAT |

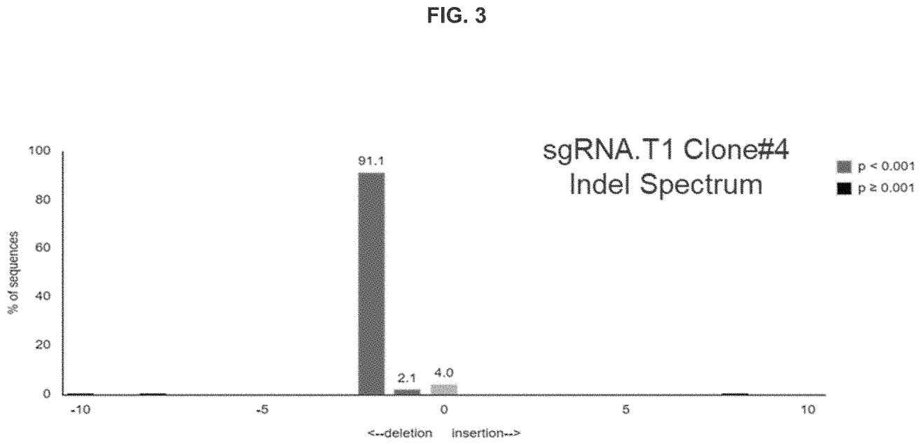

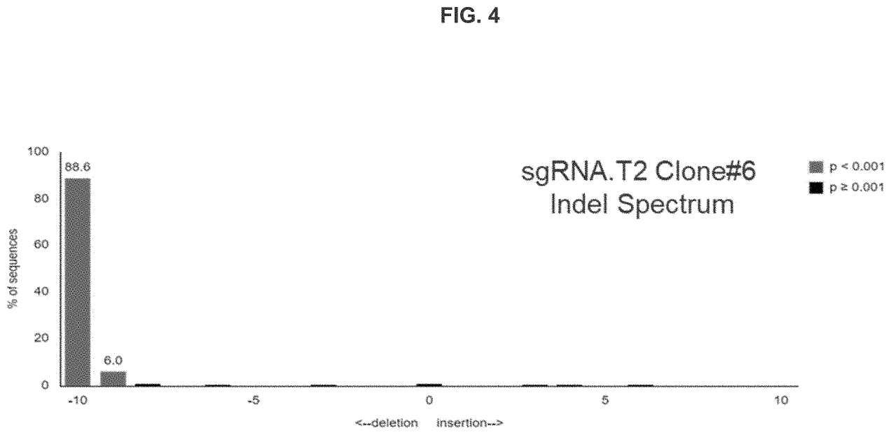

Each sgRNA was then co-transfected with the SpCas9 nuclease and AcBIVVBac.Polh.AAV2.RepTn7 bacmid DNA in Sf9 cells, seeded at 0.5×10⁶ per mL in T25 flasks in serum-free ESF-921 medium, using Cellfectin@ (Invitrogen™) transfection reagent. At 4-5 days post-transfection, cells were visualized under the fluorescence microscope and the results showed ˜10% RFP+ cells for both the sgRNA targets which suggest that the viral infection was restricted to a single cell most likely due to the mutated vp80 coding sequence. To determine the indels induced by each sgRNA, the progeny baculovirus was harvested and plaque purified in a complement Sf.39K.VP80 cell line, as described earlier (Jarvis, 2014). At 5-6 days post-infection, ten plaque purified RFP+ clones were amplified to P1 in Sf.39K.VP80 cells seeded at 0.5×106 per mL in T25 flasks in ESF-921 medium supplemented with 10% FBS. The fluorescence microscopic observation of the amplified clones showed ˜80% RFP+ cells which suggest that the Sf.39K.VP80 cell line was able to complement the VP80 function in trans required for the progeny virus production. Each clonal virus was harvested by the low-speed centrifugation and an aliquot was then used for the baculovirus DNA isolation by the Qiagen's DNeasy Blood and Tissue genomic DNA isolation kit (catalog no. 69506), according to the manufacturer's instructions. The resulting DNA was used as a template to PCR amplify each target sequence with the primers specific to the AcMNPV vp80 coding sequence. The PCR amplimers were then gel purified and directly sequenced through the Genewiz sequencing facility. The resulting sequences were analyzed by the TIDE (Tracking of Indels by DEcomposition) program (tide.deskgen.com) using default settings to determine the indels induced by each sgRNA. The TIDE analysis showed frameshift mutations in 2/10 clones for sgRNA.T1 with the highest (91%) −2 bp deletions (FIG. 3) and 1/10 clones for sgRNA.T2 with the highest (89%) −10 bp deletions in the vp80 coding sequence with no detectable insertion (FIG. 4). One of the clones of sgRNA.T1 was amplified to produce working BEV stock (Passage 2) followed by titering in Sf.39K.VP80 cells, as described earlier (Jarvis, 2014). Titrated working stock of AcBIVVBac.Polh.AAV2.Rep^{Tn7} vp80KO BEV was then used for infection in stable cell lines for ceDNA vector production.

### Example 3: Generation of Sf39K.VP80 Complement Cell Line

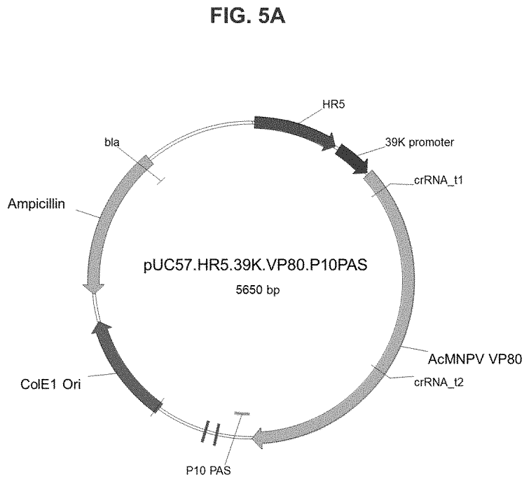

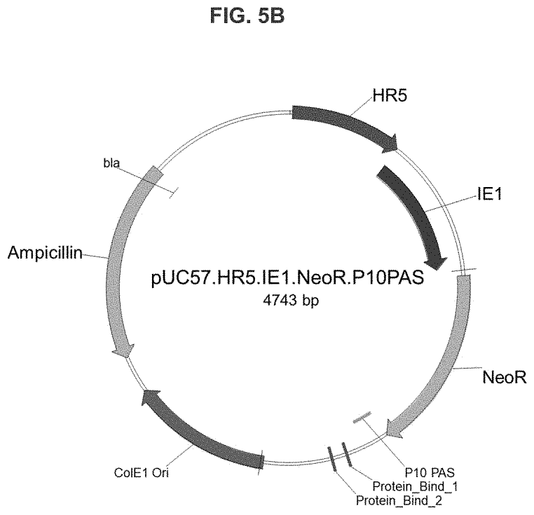

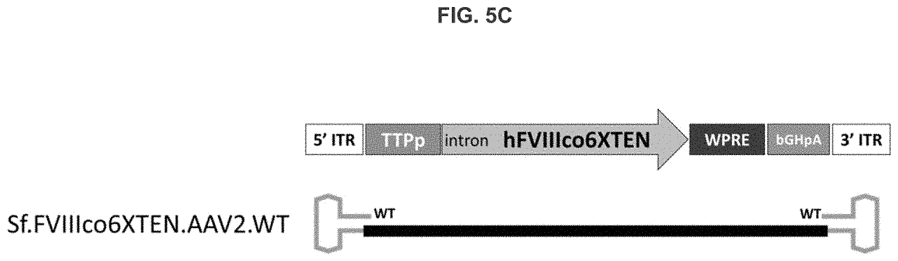

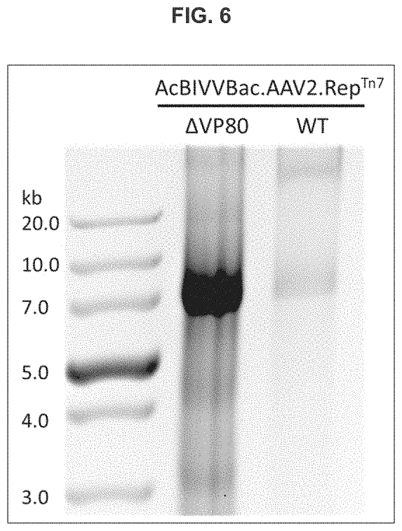

The Sf.39K.VP80 stable cell line was generated to complement the VP80 function in trans for producing the working stock of AcBIWBac.Polh.AAV2.Rep^{Tn7} vp80KO BEV. The AcMNPV-inducible 39K promoter was used for vp80 to avoid any toxic effect of VP80 over-expression on Sf9 cell growth, as observed earlier (Marek et al., 2011). To generate complement cell line, a transfer vector was produced, encoding the AcMNPV vp80 gene under the AcMNPV 39K promoter followed by the p10 polyadenylation signal (FIG. 5A). This transfer vector was then co-transfected with a plasmid encoding a neomycin resistance gene under the AcMNPV ie1 promoter preceded by the transcriptional enhancer hr5 element and followed by the p10 polyadenylation signal, as described above using Cellfectin® (Invitrogen™) transfection reagent (FIG. 5B). Also co-transfected was a plasmid encoding a hFVIIIco6XTEN expression cassette flanked by the symmetric and asymmetric ITRs of AAV (FIG. 5C).

At 24 h post-transfection, cells were visualized under the fluorescence microscope to determine the transfection efficiency and the results showed >80% GFP+ cells suggesting higher transfection efficiency. At 72 h post-transfection, cells were selected with G418 antibiotic (Sigma Aldrich) suspended in complete TNMFH medium (Grace's Insect Medium supplemented with 10% FBS+0.1% Pluronic F68) at 1.0 mg/mL final concentration. After about a week of selection, there were ˜50% of transformed cells recovered which suggests that the neomycin resistant marker was stably integrated into this cell population. The survivor cells were taken off the selection media and fed with a fresh complete TNMFH medium until confluence growth. The confluent cells were progressively expanded as an adherent culture into larger culture vessels as they continue to divide. Later, each cell line was adapted to the suspension culture by growth in shake flasks for one passage in complete TNMFH and one passage in ESF-921 medium supplemented with 10% FBS. Finally, each cell line was adapted to serum-free ESF-921 in shake flasks as suspension cultures. These shake flask cultures were routinely maintained in serum-free ESF-921 medium with passages every four days and cell growth was monitored. Finally, the polyclonal cell population of Sf.39K.VP80 cell line was used for plaque purification and amplification of the AcBIWBac.Polh.AAV2.Rep^{Tn7} vp80KO BEV, as described in Example 2.

### Example 4: Human FVIIIco6XTEN ceDNA Production Using VP80KO BEV

To determine whether the approach of using the vp80KO virus could reduce the baculoviral DNA contamination in a ceDNA preparation encoding a human FVIII transgene, a AcBIVVBac.Polh.AAV2.Rep^{Tn7} vp80KO BEV was tested in comparison with a corresponding wildtype BEV containing an intact vp80.

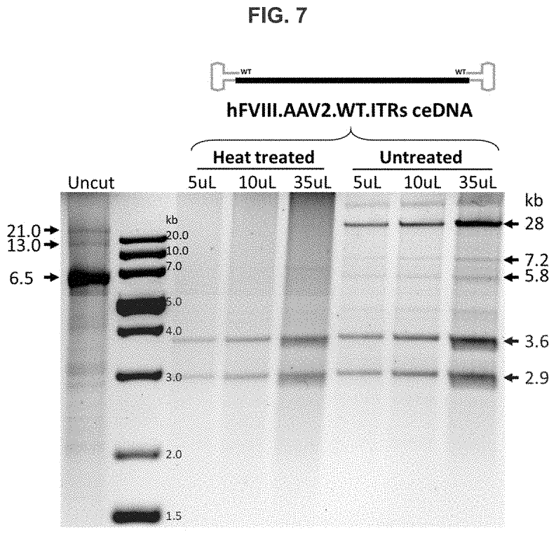

Cells were infected with the titrated working stocks of each recombinant BEV at a multiplicity of infection (MOI) of 3 pfu/cell. Cells were then gently tumbled at room temperature for 1.5 h, pelleted at 500×g for 5 min, the supernatant was aspirated, and the cells were washed once with 10 mL of fresh ESF-921 medium. The cells were then suspended into 50 mL of ESF-921 medium and then, incubated for 72 h at 28° C. in a shaker incubator. At 72 h post-infection, infected cells were harvested, and the pellets were washed once with 1×PBS to remove residual baculoviral particles and/or the culture medium. The cell pellets were then processed to purify the ceDNA vectors using the PureLink Maxi Prep DNA isolation kit (Invitrogen), according to the manufacturer's instructions. Elution fractions were analyzed by 0.8 to 1.2% agarose gel electrophoresis to determine the yield and purity of each ceDNA vector. The gel assay results showed a single thick band of the size of hFVIIIco6XTEN expression cassette (˜7.0 kb) with no detectable high molecular weight (>20 kb) baculoviral genomic DNA contamination in a vp80KO BEV infected sample in comparison with the wildtype BEV (FIG. 7). The result suggests that the vp80KO approach was able to reduce the baculoviral genomic DNA contamination and simultaneously improve the ceDNA yield. This approach was able to yield up to 0.5 mg of ceDNA vector encoding hFVIIIco6XTEN (˜7.0 kb) from 5×108 cells.

### Example 5: Human FVIIIco6XTEN ceDNA Characterization

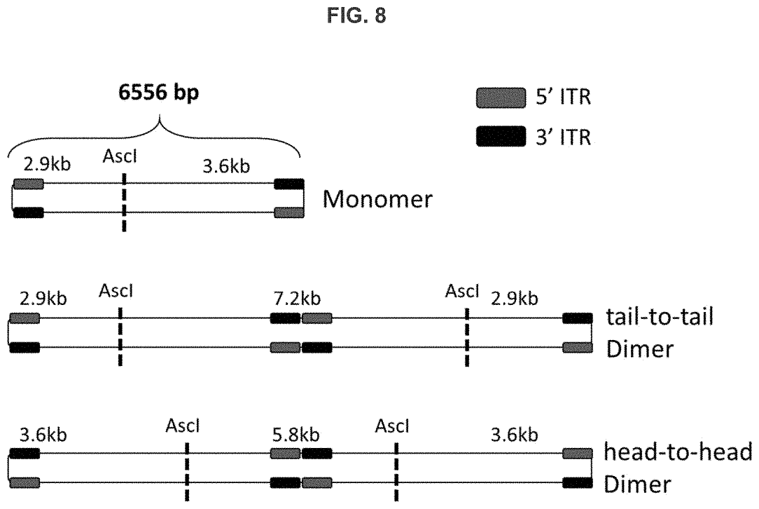

Finally, we performed a biochemical characterization of linear ITR-flanked hFVIIIco6XTEN vector DNA obtained from the stable cell line following the vp80KO virus infection. We determined whether this vector DNA has covalently closed ends, double-stranded conformation, and concatemerized multimeric forms under different conditions. First, we heat-treated the DNA (˜8.5 μg) at 95° C. for 10 min and then renatured them on ice for 30 min. Subsequently, the heat-treated or untreated vector DNAs were digested with a unique restriction endonuclease AscI, which has a single recognition site in the hFVIIIco6XTEN coding sequence. The digested samples were analyzed at different volumes on native agarose gel electrophoresis. The gel assay of the uncut vector DNA genome showed one major band of 6.5 kb and two minor bands of 13.0 kb and 21.0 kb. The 6.5 kb band was as expected for a monomeric, and the 13.0 kb and 21.0 kb bands were consistent with the dimeric or trimeric concatemerized vector genome (FIG. 7, uncut). However, for the heat-treated sample, we expected that the heat treatment would denature the DNA and could break apart the concatemerized multimeric forms except for the monomeric form, which could renature as a double-stranded DNA. Indeed, we observed that the heat-treated vector DNA, followed by AscI digestion, resolved as two major bands of 3.6 kb and 2.9 kb with no detectable high molecular weight DNA bands (FIG. 7, left panel, and FIG. 8, monomer). The 3.6 kb and 2.9 kb bands were as expected for the digested linear monomeric duplex molecule that renatured following incubation on ice. The absence of high molecular weight DNA bands was consistent with the denaturation of concatemerized multimeric forms upon heat-treatment that failed to renature following incubation on ice. In contrast, the gel analysis of AscI digested untreated vector DNA resolved as two major bands of 3.6 kb and 2.9 kb and two minor bands of 7.2 kb and 5.8 kb (FIG. 7, right panel). The 3.6 kb and 2.9 kb bands were as expected for the digestion of a vector genome monomer with AscI (FIG. 8, monomer) whereas the 7.2 kb and 5.8 kb bands were consistent with the tail-to-tail and head-to-head concatemers of multimeric vector genomes, respectively (FIG. 8, dimers). There was another major band of >20 kb observed which could be a trimeric or tetrameric form of the vector genome and difficult to explain by the simple restriction digestion analysis. Nevertheless, these results suggest that the hFVIIIco6XTEN ceDNA vector is a linear covalently closed double-stranded DNA that concatemerized in multimeric forms under native conditions.

### Sequences

TABLE 3
Additional nucleotide or amino acid sequences

| SEQ ID | |
|---|---|
| NO and | |
| Description | Nucleotide or Amino Acid Sequence |
| SEQ ID | TTGTTGTTGTACATGCGCCATCTTAGTTTTATATCAGCTGGCGCCTTAGTTATATAACATGCATGTTATATAACTAA |
| NO: 12 | GGCGCCAGCTGATATAAAACTAAGATGGCGCATGTACAACAACAACACATTAAAAGATATAGAGTTTCGCGATTGC |
| HBoV1 WT | |
| ITR 5′ | |
| SEQ ID | TATATGTGACGTGGTTGTACAGACGCCATCTTGGAATCCAATATGTCTGCCGGCGATTAGATCATGCGCGCGCGCAG |
| NO. 13 | CGCGCTGCGCGCAGCGCAGGCATGACTGAGCCGGCAGACATATTGGATTCCAAGATGGCGTCTGTACAACCAC |
| HBoV1 WT | |
| ITR 3′ | |
| SEQ ID | GGCCCCAGGTTAATTTTTAAAAAGCAGTCAAAGGTCAAAGTGGCCCTTGGCAGCATTTACTCTCTCTATTGACTTTGGTTA |
| NO: 14 | ATAATCTCAGGAGCACAAACATTCCTGGAGGCAGGAGAAGAAATCAACATCCTGGACTTATCCTCTGGGCCTCTCCCCACC |
| V2.0 | TTCGATGGCCCCAGGTTAATTTTTAAAAAGCAGTCAAAGGTCAAAGTGGCCCTTGGCAGCATTTACTCTCTCTATTGACTT |
| Expression | TGGTTAATAATCTCAGGAGCACAAACATTCCTGGAGGCAGGAGAAGAAATCAACATCCTGGACTTATCCTCTGGGCCTCTC |
| cassette | CCCACCGATATCTACCTGCTGATCGCCCGGCCCCTGTTCAAACATGTCCTAATACTCTGTCGGGGCAAAGGTCGGCAGTAG |
| mTTR482- | TTTTCCATCTTACTCAACATCCTCCCAGTGTACGTAGGATCCTGTCTGTCTGCACATTTCGTAGAGCGAGTGTTCCGATAC |
| Intron- | TCTAATCTCCCGGGGCAAAGGTCGTATTGACTTAGGTTACTTATTCTCCTTTTGTTGACTAAGTCAATAATCAGAATCAGC |
| coBDDFVIII | AGGTTTGGAGTCAGCTTGGCAGGGATCAGCAGCCTGGGTTGGAAGGAGGGGGTATAAAAGCCCCTTCACCAGGAGAAGCCG |
| XTEN | TCACACAGATCCACAAGCTCCTGCTAGGAATTCTCAGGAGCACAAACATTCCTGGAGGCAGGAGAAGAAATCAACATCCTG |
| (V2.0)- | GACTTATCCTCTGGGCCTCTCCCCACCGATATCTACCTGCTGATCGCCCGGCCCCTGTTCAAACATGTCCTAATACTCTGT |
| WPRE- | CGGGGCAAAGGTCGGCAGTAGTTTTCCATCTTACTCAACATCCTCCCAGTGTACGTAGGATCCTGTCTGTCTGCACATTTC |
| bGHPolyA | GTAGAGCGAGTGTTCCGATACTCTAATCTCCCGGGGCAAAGGTCGTATTGACTTAGGTTACTTATTCTCCTTTTGTTGACT |
| | AAGTCAATAATCAGAATCAGCAGGTTTGGAGTCAGCTTGGCAGGGATCAGCAGCCTGGGTTGGAAGGAGGGGGTATAAAAG |
| | CCCCTTCACCAGGAGAAGCCGTCACACAGATCCACAAGCTCCTGCTAGAGTCGCTGCGCGCTGCCTTCGCCCCGTGCCCCG |
| | CTCCGCCGCCGCCTCGCGCCGCCCGCCCCGGCTCTGACTGACCGCGTTACTCCCACAGGTGAGCGGGCGGGACGGCCCTTC |
| | TCCTCCGGGCTGTAATTAGCGCTTGGTTTATTGACGGCTTGTTTCTTTTCTGTGGCTGCGTGAAAGCCTTGAGGGGCTCCG |
| | GGAAGGCCCTTTGTGCGGGGGGAGCGGCTCGGGGGGTGCGTGCGTGTGTGTGTGCGTGGGGAGCGCCGCGTGCGGCTCCGC |
| | GCTGCCCGGCGGCTGTGAGCGCTGCGGGCGCGGCGCGGGGCTTTGTGCGCTCCGCAGTGTGCGCGAGGGGAGCGCGGCCGG |
| | GGGCGGTGCCCCGCGGTGCGGGGGGGGCTGCGAGGGGAACAAAGGCTGCGTGCGGGGTGTGTGCGTGGGGGGGTGAGCAGG |
| | GGGTGTGGGCGCGTCGGTCGGGCTGCAACCCCCCCTGCACCCCCCTCCCCGAGTTGCTGAGCACGGCCCGGCTTCGGGTGC |
| | GGGGCTCCGTACGGGGCGTGGCGCGGGGCTCGCCGTGCCGGGCGGGGGGTGGCGGCAGGTGGGGGTGCCGGGCGGGGCGGG |
| | GCCGCCTCGGGCCGGGGAGGGCTCGGGGGAGGGGCGCGGCGGCCCCCGGAGCGCCGGCGGCTGTCGAGGCGCGGCGAGCCG |
| | CAGCCATTGCCTTTTATGGTAATCGTGCGAGAGGGCGCAGGGACTTCCTTTGTCCCAAATCTGTGCGGAGCCGAAATCTGG |
| | GAGGCGCCGCCGCACCCCCTCTAGCGGGCGCGGGGCGAAGCGGTGCGGCGCCGGCAGGAAGGAAATGGGCGGGGAGGGCCT |
| | TCGTGCGTCGCCGCGCCGCCGTCCCCTTCTCCCTCTCCAGCCTCGGGGCTGTCCGCGGGGGGACGGCTGCCTTCGGGGGGG |
| | ACGGGGCAGGGCGGGGTTCGGCTTCTGGCGTGTGACCGGCGGCTCTAGAGCCTCTGCTAACCTTGTTCTTGCCTTCTTCTT |
| | TTTCCTACAGCTCCTGGGCAACGTGCTGGTTATTGTGCTGTCTCATCATTTTGGCAAAGAATTACTCGAGGCCACCATGCA |
| | GATTGAACTGTCCACTTGCTTCTTCCTGTGCCTCCTGCGGTTTTGCTTCTCGGCCACCCGCCGGTATTACTTAGGTGCTGT |
| | GGAACTGAGCTGGGACTACATGCAGTCCGACCTGGGAGAACTGCCGGTGGACGCGAGATTCCCACCTAGAGTCCCGAAGTC |
| | CTTCCCATTCAACACCTCCGTGGTCTACAAAAAGACCCTGTTCGTGGAGTTCACTGACCACCTTTTCAATATTGCCAAGCC |
| | GCGCCCCCCCTGGATGGGCCTGCTTGGTCCTACGATCCAAGCAGAGGTCTACGACACCGTGGTCATCACACTGAAGAACAT |
| | GGCCTCACACCCCGTGTCGCTGCATGCTGTGGGAGTGTCCTACTGGAAGGCCTCAGAGGGTGCCGAATATGATGACCAGAC |
| | CAGCCAGAGGGAAAAGGAGGATGACAAAGTGTTCCCGGGTGGCAGCCACACTTACGTGTGGCAAGTGCTGAAGGAAAACGG |
| | GCCTATGGCGTCGGACCCCCTATGCCTGACCTACTCCTACCTGTCCCATGTGGACCTTGTGAAGGATCTCAACTCGGGACT |
| | GATCGGCGCCCTCTTGGTGTGCAGAGAAGGCAGCCTGGCGAAGGAAAAGACTCAGACCCTGCACAAGTTCATTCTGTTGTT |
| | TGCTGTGTTCGATGAAGGAAAGTCCTGGCACTCAGAAACCAAGAACTCGCTGATGCAGGATAGAGATGCGGCCTCGGCCAG |
| | AGCCTGGCCTAAAATGCACACCGTCAACGGATATGTGAACAGGTCGCTCCCTGGCCTCATCGGCTGCCACAGAAAGTCCGT |
| | GTATTGGCATGTGATCGGCATGGGTACTACTCCGGAAGTGCATAGTATCTTTCTGGAGGGCCATACCTTCTTGGTGCGCAA |
| | CCACAGACAGGCCTCGCTGGAAATCTCGCCTATCACTTTCTTGACTGCGCAGACCCTCCTTATGGACCTTGGACAGTTCCT |
| | GCTGTTCTGTCACATCAGCTCCCATCAGCATGATGGGATGGAGGCCTATGTCAAAGTGGACTCCTGCCCTGAGGAGCCACA |
| | GCTCCGGATGAAGAACAATGAGGAAGCGGAGGATTACGACGACGACCTGACTGACAGCGAAATGGACGTCGTGCGATTCGA |
| | TGACGACAACAGCCCGTCCTTCATCCAAATTAGATCAGTGGCGAAGAAGCACCCCAAGACCTGGGTGCACTACATTGCCGC |
| | CGAGGAAGAGGACTGGGACTACGCGCCGCTGGTGCTGGCGCCAGACGACAGGAGCTACAAGTCCCAGTACCTCAACAACGG |
| | GCCGCAGCGCATTGGCAGGAAGTACAAGAAAGTCCGCTTCATGGCCTACACTGATGAAACCTTCAAGACGAGGGAAGCCAT |
| | CCAGCACGAGTCAGGCATCCTGGGACCGCTCCTTTACGGCGAAGTCGGGGATACCCTGCTCATCATTTTCAAGAACCAGGC |
| | ATCGCGGCCCTACAACATCTACCCTCACGGGATCACAGACGTGCGCCCGCTCTACTCCCGCCGGCTGCCCAAGGGAGTGAA |
| | GCACCTGAAGGATTTTCCCATCCTGCCGGGAGAAATCTTCAAGTACAAGTGGACCGTGACTGTGGAAGATGGCCCTACCAA |
| | GTCGGACCCTCGCTGTCTGACCCGGTACTATTCCTCGTTTGTGAACATGGAGCGCGACCTGGCCTCGGGGCTGATTGGTCC |
| | GCTGCTGATCTGCTACAAGGAGTCCGTGGACCAGCGCGGGAACCAGATCATGTCCGACAAGCGCAACGTGATCCTGTTCTC |
| | TGTCTTTGATGAAAACAGATCGTGGTACTTGACTGAGAATATCCAGCGGTTCCTGCCCAACCCAGCGGGAGTGCAACTGGA |
| | GGACCCGGAGTTCCAGGCCTCAAACATTATGCACTCTATCAACGGCTATGTGTTCGACTCGCTCCAACTGAGCGTGTGCCT |
| | GCATGAAGTGGCATACTGGTACATTCTGTCCATCGGAGCCCAGACCGACTTCCTGTCCGTGTTCTTCTCCGGATACACCTT |
| | CAAGCATAAGATGGTGTACGAGGACACTCTGACCCTCTTCCCATTTTCCGGAGAAACTGTGTTCATGTCAATGGAAAACCC |
| | GGGCTTGTGGATTCTGGGTTGCCATAACTCGGACTTCCGGAATAGAGGGATGACCGCCCTGCTGAAAGTGTCCAGCTGTGA |
| | CAAGAATACCGGCGATTACTACGAGGACAGCTATGAGGACATCTCCGCTTATCTGCTGTCCAAGAACAACGCCATTGAACC |
| | CAGGTCCTTCTCCCAAAACGGTGCACCGACCTCCGAAAGCGCCACCCCAGAGTCAGGACCTGGCTCGGAACCGGCTACCTC |
| | GGGCTCAGAGACACCGGGGACTTCCGAGTCCGCAACCCCCGAGAGTGGACCCGGATCCGAACCAGCAACCTCAGGATCAGA |
| | AACCCCGGGAACTTCGGAATCCGCCACTCCCGAGTCGGGACCAGGCACCTCCACTGAGCCTTCCGAGGGAAGCGCCCCCGG |
| | ATCCCCTGCTGGATCCCCTACCAGCACTGAAGAAGGCACCTCAGAATCCGCGACCCCTGAGTCCGGCCCTGGAAGCGAACC |
| | CGCCACCTCCGGTTCCGAAACCCCTGGGACTAGCGAGAGCGCCACTCCGGAATCGGGCCCAGGAAGCCCTGCCGGATCCCC |
| | GACCAGCACCGAGGAGGGAAGCCCCGCCGGGTCACCGACTTCCACTGAGGAGGGAGCCTCATCCCCCCCCGTGCTGAAGCG |
| | GCATCAAAGAGAGATCACCAGGACCACTCTCCAGTCCGATCAGGAAGAAATTGACTACGACGATACTATCAGCGTGGAGAT |
| | GAAGAAGGAGGACTTCGACATCTACGATGAGGATGAGAACCAGTCCCCTCGGAGCTTTCAGAAGAAAACCCGCCACTACTT |
| | CATCGCTGCCGTGGAGCGGCTGTGGGATTACGGGATGTCCAGCTCACCGCATGTGCTGCGGAATAGAGCGCAGTCAGGATC |
| | GGTGCCCCAGTTCAAGAAGGTCGTGTTCCAAGAGTTCACCGACGGGTCCTTCACTCAACCCCTGTACCGGGGCGAACTCAA |
| | CGAACACCTGGGACTGCTTGGGCCGTATATCAGGGCAGAAGTGGAAGATAACATCATGGTCACCTTCCGCAACCAGGCCTC |
| | CCGGCCGTACAGCTTCTACTCTTCACTGATCTCCTACGAGGAAGATCAGCGGCAGGGAGCCGAGCCCCGGAAGAACTTCGT |
| | CAAGCCTAACGAAACTAAGACCTACTTTTGGAAGGTCCAGCATCACATGGCCCCGACCAAAGACGAGTTCGACTGTAAAGC |
| | CTGGGCCTACTTCTCCGATGTGGACCTGGAGAAGGACGTGCACTCGGGACTCATTGGCCCGCTCCTTGTGTGCCATACTAA |
| | TACCCTGAACCCTGCTCACGGTCGCCAAGTCACAGTGCAGGAGTTCGCCCTCTTCTTCACCATCTTCGATGAAACAAAGTC |
| | CTGGTACTTTACTGAGAACATGGAACGCAATTGCAGGGCACCCTGCAACATCCAGATGGAAGATCCCACCTTCAAGGAAAA |
| | CTACCGGTTTCATGCCATTAACGGCTACATAATGGACACGTTGCCAGGACTGGTCATGGCCCAGGACCAGAGAATCCGGTG |
| | GTATCTGCTCTCCATGGGCTCCAACGAAAACATTCACAGCATTCATTTTTCCGGCCATGTGTTCACCGTCCGGAAGAAGGA |
| | AGAGTACAAGATGGCTCTGTACAACCTCTACCCTGGAGTGTTCGAGACTGTGGAAATGCTGCCTAGCAAGGCCGGCATTTG |
| | GAGAGTGGAATGCCTGATCGGAGAGCATTTGCACGCCGGAATGTCCACCCTGTTTCTTGTGTACTCCAACAAGTGCCAGAC |
| | CCCGCTGGGAATGGCCTCAGGTCATATTAGGGATTTCCAGATCACTGCTTCGGGGCAGTACGGGCAGTGGGCACCTAAGTT |
| | GGCCCGGCTGCACTACTCTGGCTCCATCAATGCCTGGTCCACCAAGGAACCCTTCTCCTGGATTAAGGTGGACCTCCTGGC |
| | CCCAATGATTATTCACGGTATTAAGACCCAGGGTGCCCGACAGAAGTTCTCCTCACTCTACATCTCGCAATTCATCATAAT |
| | GTACAGCCTGGATGGGAAGAAGTGGCAGACCTACCGGGGAAACTCCACTGGAACGCTCATGGTGTTTTTCGGCAACGTGGA |
| | CTCCTCCGGCATTAAGCACAACATCTTCAACCCTCCGATCATTGCTCGGTACATCCGGCTGCACCCAACTCACTACAGCAT |
| | CCGGTCCACCCTGCGGATGGAACTGATGGGTTGTGACCTGAACTCCTGCTCCATGCCCCTTGGGATGGAATCCAAGGCCAT |
| | TAGCGATGCACAGATCACCGCCTCTTCATACTTCACCAACATGTTCGCGACCTGGTCCCCGTCGAAGGCCCGCCTGCACCT |
| | CCAAGGTCGCTCCAATGCGTGGCGGCCTCAAGTGAACAACCCCAAGGAGTGGCTCCAGGTCGACTTCCAAAAGACCATGAA |
| | GGTCACCGGAGTGACCACCCAGGGCGTGAAGTCCCTGCTGACCTCTATGTACGTTAAGGAGTTCCTCATCTCCTCAAGCCA |
| | AGACGGACATCAGTGGACCCTGTTCTTCCAAAACGGAAAAGTCAAAGTATTCCAGGGCAACCAGGACTCCTTCACCCCTGT |
| | GGTCAACAGCCTGGACCCCCCATTGCTGACCCGCTACCTCCGCATCCACCCCCAAAGCTGGGTCCACCAGATCGCACTGCG |
| | CATGGAGGTCCTTGGATGCGAAGCCCAAGATCTGTACTAAGCGGCCGCTCATAATCAACCTCTGGATTACAAAATTTGTGA |
| | AAGATTGACTGGTATTCTTAACTATGTTGCTCCTTTTACGCTATGTGGATACGCTGCTTTAATGCCTTTGTATCATGCTAT |
| | TGCTTCCCGTATGGCTTTCATTTTCTCCTCCTTGTATAAATCCTGGTTGCTGTCTCTTTATGAGGAGTTGTGGCCCGTTGT |
| | CAGGCAACGTGGCGTGGTGTGCACTGTGTTTGCTGACGCAACCCCCACTGGTTGGGGCATTGCCACCACCTGTCAGCTCCT |
| | TTCCGGGACTTTCGCTTTCCCCCTCCCTATTGCCACGGCGGAACTCATCGCCGCCTGCCTTGCCCGCTGCTGGACAGGGGC |
| | TCGGCTGTTGGGCACTGACAATTCCGTGGTGTTGTCGGGGAAATCATCGTCCTTTCCTTGGCTGCTCGCCTGTGTTGCCAC |
| | CTGGATTCTGCGCGGGACGTCCTTCTGCTACGTCCCTTCGGCCCTCAATCCAGCGGACCTTCCTTCCCGCGGCCTGCTGCC |
| | GGCTCTGCGGCCTCTTCCGCGTCTTCGCCTTCGCCCTCAGACGAGTCGGATCTCCCTTTGGGCCGCCTCCCCGCTGCCTAG |
| | GCGACTGTGCCTTCTAGTTGCCAGCCATCTGTTGTTTGCCCCTCCCCCGTGCCTTCCTTGACCCTGGAAGGTGCCACTCCC |
| | ACTGTCCTTTCCTAATAAAATGAGGAAATTGCATCGCATTGTCTGAGTAGGTGTCATTCTATTCTGGGGGGTGGGGTGGGG |
| | CAGGACAGCAAGGGGGAGGATTGGGAAGACAATAGCAGGCATGCTGGGGAAGACCATGGGCGCGCCAGGCCTGTCGACGCC |
| | CGGGCGGTACCGCGATCGCTCGCGACGCATAAAG |
| SEQ ID | ATGCAGATTGAACTGTCCACTTGCTTCTTCCTGTGCCTCCTGCGGTTTTGCTTCTCGGCCACCCGCCGGTATTACTTAGGT |
| NO: 15 | GCTGTGGAACTGAGCTGGGACTACATGCAGTCCGACCTGGGAGAACTGCCGGTGGACGCGAGATTCCCACCTAGAGTCCCG |
| Nucleotide | AAGTCCTTCCCATTCAACACCTCCGTGGTCTACAAAAAGACCCTGTTCGTGGAGTTCACTGACCACCTTTTCAATATTGCC |
| sequence | AAGCCGCGCCCCCCCTGGATGGGCCTGCTTGGTCCTACGATCCAAGCAGAGGTCTACGACACCGTGGTCATCACACTGAAG |
| encoding | AACATGGCCTCACACCCCGTGTCGCTGCATGCTGTGGGAGTGTCCTACTGGAAGGCCTCAGAGGGTGCCGAATATGATGAC |
| coBDDFVIII | CAGACCAGCCAGAGGGAAAAGGAGGATGACAAAGTGTTCCCGGGTGGCAGCCACACTTACGTGTGGCAAGTGCTGAAGGAA |
| XTEN | AACGGGCCTATGGCGTCGGACCCCCTATGCCTGACCTACTCCTACCTGTCCCATGTGGACCTTGTGAAGGATCTCAACTCG |
| (V2.0) | GGACTGATCGGCGCCCTCTTGGTGTGCAGAGAAGGCAGCCTGGCGAAGGAAAAGACTCAGACCCTGCACAAGTTCATTCTG |
| | TTGTTTGCTGTGTTCGATGAAGGAAAGTCCTGGCACTCAGAAACCAAGAACTCGCTGATGCAGGATAGAGATGCGGCCTCG |
| | GCCAGAGCCTGGCCTAAAATGCACACCGTCAACGGATATGTGAACAGGTCGCTCCCTGGCCTCATCGGCTGCCACAGAAAG |
| | TCCGTGTATTGGCATGTGATCGGCATGGGTACTACTCCGGAAGTGCATAGTATCTTTCTGGAGGGCCATACCTTCTTGGTG |
| | CGCAACCACAGACAGGCCTCGCTGGAAATCTCGCCTATCACTTTCTTGACTGCGCAGACCCTCCTTATGGACCTTGGACAG |
| | TTCCTGCTGTTCTGTCACATCAGCTCCCATCAGCATGATGGGATGGAGGCCTATGTCAAAGTGGACTCCTGCCCTGAGGAG |
| | CCACAGCTCCGGATGAAGAACAATGAGGAAGCGGAGGATTACGACGACGACCTGACTGACAGCGAAATGGACGTCGTGCGA |
| | TTCGATGACGACAACAGCCCGTCCTTCATCCAAATTAGATCAGTGGCGAAGAAGCACCCCAAGACCTGGGTGCACTACATT |
| | GCCGCCGAGGAAGAGGACTGGGACTACGCGCCGCTGGTGCTGGCGCCAGACGACAGGAGCTACAAGTCCCAGTACCTCAAC |
| | AACGGGCCGCAGCGCATTGGCAGGAAGTACAAGAAAGTCCGCTTCATGGCCTACACTGATGAAACCTTCAAGACGAGGGAA |
| | GCCATCCAGCACGAGTCAGGCATCCTGGGACCGCTCCTTTACGGCGAAGTCGGGGATACCCTGCTCATCATTTTCAAGAAC |
| | CAGGCATCGCGGCCCTACAACATCTACCCTCACGGGATCACAGACGTGCGCCCGCTCTACTCCCGCCGGCTGCCCAAGGGA |
| | GTGAAGCACCTGAAGGATTTTCCCATCCTGCCGGGAGAAATCTTCAAGTACAAGTGGACCGTGACTGTGGAAGATGGCCCT |
| | ACCAAGTCGGACCCTCGCTGTCTGACCCGGTACTATTCCTCGTTTGTGAACATGGAGCGCGACCTGGCCTCGGGGCTGATT |
| | GGTCCGCTGCTGATCTGCTACAAGGAGTCCGTGGACCAGCGCGGGAACCAGATCATGTCCGACAAGCGCAACGTGATCCTG |
| | TTCTCTGTCTTTGATGAAAACAGATCGTGGTACTTGACTGAGAATATCCAGCGGTTCCTGCCCAACCCAGCGGGAGTGCAA |
| | CTGGAGGACCCGGAGTTCCAGGCCTCAAACATTATGCACTCTATCAACGGCTATGTGTTCGACTCGCTCCAACTGAGCGTG |
| | TGCCTGCATGAAGTGGCATACTGGTACATTCTGTCCATCGGAGCCCAGACCGACTTCCTGTCCGTGTTCTTCTCCGGATAC |
| | ACCTTCAAGCATAAGATGGTGTACGAGGACACTCTGACCCTCTTCCCATTTTCCGGAGAAACTGTGTTCATGTCAATGGAA |
| | AACCCGGGCTTGTGGATTCTGGGTTGCCATAACTCGGACTTCCGGAATAGAGGGATGACCGCCCTGCTGAAAGTGTCCAGC |
| | TGTGACAAGAATACCGGCGATTACTACGAGGACAGCTATGAGGACATCTCCGCTTATCTGCTGTCCAAGAACAACGCCATT |
| | GAACCCAGGTCCTTCTCCCAAAACGGTGCACCGACCTCCGAAAGCGCCACCCCAGAGTCAGGACCTGGCTCGGAACCGGCT |
| | ACCTCGGGCTCAGAGACACCGGGGACTTCCGAGTCCGCAACCCCCGAGAGTGGACCCGGATCCGAACCAGCAACCTCAGGA |
| | TCAGAAACCCCGGGAACTTCGGAATCCGCCACTCCCGAGTCGGGACCAGGCACCTCCACTGAGCCTTCCGAGGGAAGCGCC |
| | CCCGGATCCCCTGCTGGATCCCCTACCAGCACTGAAGAAGGCACCTCAGAATCCGCGACCCCTGAGTCCGGCCCTGGAAGC |
| | GAACCCGCCACCTCCGGTTCCGAAACCCCTGGGACTAGCGAGAGCGCCACTCCGGAATCGGGCCCAGGAAGCCCTGCCGGA |
| | TCCCCGACCAGCACCGAGGAGGGAAGCCCCGCCGGGTCACCGACTTCCACTGAGGAGGGAGCCTCATCCCCCCCCGTGCTG |
| | AAGCGGCATCAAAGAGAGATCACCAGGACCACTCTCCAGTCCGATCAGGAAGAAATTGACTACGACGATACTATCAGCGTG |
| | GAGATGAAGAAGGAGGACTTCGACATCTACGATGAGGATGAGAACCAGTCCCCTCGGAGCTTTCAGAAGAAAACCCGCCAC |
| | TACTTCATCGCTGCCGTGGAGCGGCTGTGGGATTACGGGATGTCCAGCTCACCGCATGTGCTGCGGAATAGAGCGCAGTCA |
| | GGATCGGTGCCCCAGTTCAAGAAGGTCGTGTTCCAAGAGTTCACCGACGGGTCCTTCACTCAACCCCTGTACCGGGGCGAA |
| | CTCAACGAACACCTGGGACTGCTTGGGCCGTATATCAGGGCAGAAGTGGAAGATAACATCATGGTCACCTTCCGCAACCAG |
| | GCCTCCCGGCCGTACAGCTTCTACTCTTCACTGATCTCCTACGAGGAAGATCAGCGGCAGGGAGCCGAGCCCCGGAAGAAC |
| | TTCGTCAAGCCTAACGAAACTAAGACCTACTTTTGGAAGGTCCAGCATCACATGGCCCCGACCAAAGACGAGTTCGACTGT |
| | AAAGCCTGGGCCTACTTCTCCGATGTGGACCTGGAGAAGGACGTGCACTCGGGACTCATTGGCCCGCTCCTTGTGTGCCAT |
| | ACTAATACCCTGAACCCTGCTCACGGTCGCCAAGTCACAGTGCAGGAGTTCGCCCTCTTCTTCACCATCTTCGATGAAACA |
| | AAGTCCTGGTACTTTACTGAGAACATGGAACGCAATTGCAGGGCACCCTGCAACATCCAGATGGAAGATCCCACCTTCAAG |
| | GAAAACTACCGGTTTCATGCCATTAACGGCTACATAATGGACACGTTGCCAGGACTGGTCATGGCCCAGGACCAGAGAATC |
| | CGGTGGTATCTGCTCTCCATGGGCTCCAACGAAAACATTCACAGCATTCATTTTTCCGGCCATGTGTTCACCGTCCGGAAG |
| | AAGGAAGAGTACAAGATGGCTCTGTACAACCTCTACCCTGGAGTGTTCGAGACTGTGGAAATGCTGCCTAGCAAGGCCGGC |
| | ATTTGGAGAGTGGAATGCCTGATCGGAGAGCATTTGCACGCCGGAATGTCCACCCTGTTTCTTGTGTACTCCAACAAGTGC |
| | CAGACCCCGCTGGGAATGGCCTCAGGTCATATTAGGGATTTCCAGATCACTGCTTCGGGGCAGTACGGGCAGTGGGCACCT |
| | AAGTTGGCCCGGCTGCACTACTCTGGCTCCATCAATGCCTGGTCCACCAAGGAACCCTTCTCCTGGATTAAGGTGGACCTC |
| | CTGGCCCCAATGATTATTCACGGTATTAAGACCCAGGGTGCCCGACAGAAGTTCTCCTCACTCTACATCTCGCAATTCATC |
| | ATAATGTACAGCCTGGATGGGAAGAAGTGGCAGACCTACCGGGGAAACTCCACTGGAACGCTCATGGTGTTTTTCGGCAAC |
| | GTGGACTCCTCCGGCATTAAGCACAACATCTTCAACCCTCCGATCATTGCTCGGTACATCCGGCTGCACCCAACTCACTAC |
| | AGCATCCGGTCCACCCTGCGGATGGAACTGATGGGTTGTGACCTGAACTCCTGCTCCATGCCCCTTGGGATGGAATCCAAG |
| | GCCATTAGCGATGCACAGATCACCGCCTCTTCATACTTCACCAACATGTTCGCGACCTGGTCCCCGTCGAAGGCCCGCCTG |
| | CACCTCCAAGGTCGCTCCAATGCGTGGCGGCCTCAAGTGAACAACCCCAAGGAGTGGCTCCAGGTCGACTTCCAAAAGACC |
| | ATGAAGGTCACCGGAGTGACCACCCAGGGCGTGAAGTCCCTGCTGACCTCTATGTACGTTAAGGAGTTCCTCATCTCCTCA |
| | AGCCAAGACGGACATCAGTGGACCCTGTTCTTCCAAAACGGAAAAGTCAAAGTATTCCAGGGCAACCAGGACTCCTTCACC |
| | CCTGTGGTCAACAGCCTGGACCCCCCATTGCTGACCCGCTACCTCCGCATCCACCCCCAAAGCTGGGTCCACCAGATCGCA |
| | CTGCGCATGGAGGTCCTTGGATGCGAAGCCCAAGATCTGTACTAA |
| SEQ ID | GGCCCCAGGTTAATTTTTAAAAAGCAGTCAAAGGTCAAAGTGGCCCTTGGCAGCATTTACTCTCTCTATTGACTTTGGTTA |
| NO: 16 | ATAATCTCAGGAGCACAAACATTCCTGGAGGCAGGAGAAGAAATCAACATCCTGGACTTATCCTCTGGGCCTCTCCCCACC |
| A1MB2 | TTCGATGGCCCCAGGTTAATTTTTAAAAAGCAGTCAAAGGTCAAAGTGGCCCTTGGCAGCATTTACTCTCTCTATTGACTT |
| enhancer | TGGTTAATAATCTCAGGAGCACAAACATTCCTGGAGGCAGGAGAAGAAATCAACATCCTGGACTTATCCTCTGGGCCTCTC |
| | CCCACC |
| SEQ ID | GATATCTACCTGCTGATCGCCCGGCCCCTGTTCAAACATGTCCTAATACTCTGTCGGGGCAAAGGTCGGCAGTAGTTTTCC |
| NO: 17 | ATCTTACTCAACATCCTCCCAGTGTACGTAGGATCCTGTCTGTCTGCACATTTCGTAGAGCGAGTGTTCCGATACTCTAAT |
| mTTR | CTCCCGGGGCAAAGGTCGTATTGACTTAGGTTACTTATTCTCCTTTTGTTGACTAAGTCAATAATCAGAATCAGCAGGTTT |
| promoter | GGAGTCAGCTTGGCAGGGATCAGCAGCCTGGGTTGGAAGGAGGGGGTATAAAAGCCCCTTCACCAGGAGAAGCCGTCACAC |
| | AGATCCACAAGCTCCTGCTAG |
| SEQ ID | TCAGGAGCACAAACATTCCTGGAGGCAGGAGAAGAAATCAACATCCTGGACTTATCCTCTGGGCCTCTCCCCACCGA |
| NO: 18 | TATCTACCTGCTGATCGCCCGGCCCCTGTTCAAACATGTCCTAATACTCTGTCGGGGCAAAGGTCGGCAGTAGTTTT |
| Chimeric | CCATCTTACTCAACATCCTCCCAGTGTACGTAGGATCCTGTCTGTCTGCACATTTCGTAGAGCGAGTGTTCCGATAC |
| Intron | TCTAATCTCCCGGGGCAAAGGTCGTATTGACTTAGGTTACTTATTCTCCTTTTGTTGACTAAGTCAATAATCAGAAT |
| | CAGCAGGTTTGGAGTCAGCTTGGCAGGGATCAGCAGCCTGGGTTGGAAGGAGGGGGTATAAAAGCCCCTTCACCAGG |
| | AGAAGCCGTCACACAGATCCACAAGCTCCTGCTAGAGTCGCTGCGCGCTGCCTTCGCCCCGTGCCCCGCTCCGCCGC |
| | CGCCTCGCGCCGCCCGCCCCGGCTCTGACTGACCGCGTTACTCCCACAGGTGAGCGGGCGGGACGGCCCTTCTCCTC |
| | CGGGCTGTAATTAGCGCTTGGTTTATTGACGGCTTGTTTCTTTTCTGTGGCTGCGTGAAAGCCTTGAGGGGCTCCGG |
| | GAAGGCCCTTTGTGCGGGGGGAGCGGCTCGGGGGGTGCGTGCGTGTGTGTGTGCGTGGGGAGCGCCGCGTGCGGCTC |
| | CGCGCTGCCCGGCGGCTGTGAGCGCTGCGGGCGCGGCGCGGGGCTTTGTGCGCTCCGCAGTGTGCGCGAGGGGAGCG |
| | CGGCCGGGGGCGGTGCCCCGCGGTGCGGGGGGGGCTGCGAGGGGAACAAAGGCTGCGTGCGGGGTGTGTGCGTGGGG |
| | GGGTGAGCAGGGGGTGTGGGCGCGTCGGTCGGGCTGCAACCCCCCCTGCACCCCCCTCCCCGAGTTGCTGAGCACGG |
| | CCCGGCTTCGGGTGCGGGGCTCCGTACGGGGCGTGGCGCGGGGCTCGCCGTGCCGGGCGGGGGGTGGCGGCAGGTGG |
| | GGGTGCCGGGCGGGGCGGGGCCGCCTCGGGCCGGGGAGGGCTCGGGGGAGGGGCGCGGCGGCCCCCGGAGCGCCGGC |
| | GGCTGTCGAGGCGCGGCGAGCCGCAGCCATTGCCTTTTATGGTAATCGTGCGAGAGGGCGCAGGGACTTCCTTTGTC |
| | CCAAATCTGTGCGGAGCCGAAATCTGGGAGGCGCCGCCGCACCCCCTCTAGCGGGCGCGGGGCGAAGCGGTGCGGCG |
| | CCGGCAGGAAGGAAATGGGCGGGGAGGGCCTTCGTGCGTCGCCGCGCCGCCGTCCCCTTCTCCCTCTCCAGCCTCGG |
| | GGCTGTCCGCGGGGGGACGGCTGCCTTCGGGGGGGACGGGGCAGGGCGGGGTTCGGCTTCTGGCGTGTGACCGGCGG |
| | CTCTAGAGCCTCTGCTAACCTTGTTCTTGCCTTCTTCTTTTTCCTACAGCTCCTGGGCAACGTGCTGGTTATTGTGC |
| | TGTCTCATCATTTTGGCAAAGAATTA |
| SEQ ID | TCATAATCAACCTCTGGATTACAAAATTTGTGAAAGATTGACTGGTATTCTTAACTATGTTGCTCCTTTTACGCTATGTGG |
| NO: 19 | ATACGCTGCTTTAATGCCTTTGTATCATGCTATTGCTTCCCGTATGGCTTTCATTTTCTCCTCCTTGTATAAATCCTGGTT |
| WPRE | GCTGTCTCTTTATGAGGAGTTGTGGCCCGTTGTCAGGCAACGTGGCGTGGTGTGCACTGTGTTTGCTGACGCAACCCCCAC |
| | TGGTTGGGGCATTGCCACCACCTGTCAGCTCCTTTCCGGGACTTTCGCTTTCCCCCTCCCTATTGCCACGGCGGAACTCAT |
| | CGCCGCCTGCCTTGCCCGCTGCTGGACAGGGGCTCGGCTGTTGGGCACTGACAATTCCGTGGTGTTGTCGGGGAAATCATC |
| | GTCCTTTCCTTGGCTGCTCGCCTGTGTTGCCACCTGGATTCTGCGCGGGACGTCCTTCTGCTACGTCCCTTCGGCCCTCAA |
| | TCCAGCGGACCTTCCTTCCCGCGGCCTGCTGCCGGCTCTGCGGCCTCTTCCGCGTCTTCGCCTTCGCCCTCAGACGAGTCG |
| | GATCTCCCTTTGGGCCGCCTCCCCGCTG |
| SEQ ID | CGACTGTGCCTTCTAGTTGCCAGCCATCTGTTGTTTGCCCCTCCCCCGTGCCTTCCTTGACCCTGGAAGGTGCCACT |
| NO: 20 | CCCACTGTCCTTTCCTAATAAAATGAGGAAATTGCATCGCATTGTCTGAGTAGGTGTCATTCTATTCTGGGGGGTGG |
| bGHpA | GGTGGGGCAGGACAGCAAGGGGGAGGATTGGGAAGACAATAGCAGGCATGCTGGGGA |
| SEQ ID | GCCACTCGCCGGTACTACCTTGGAGCCGTGGAGCTTTCATGGGACTACATGCAGAGCGACCTGGGCGAACTCCCCGT |
| NO: 21 | GGATGCCAGATTCCCCCCCCGCGTGCCAAAGTCCTTCCCCTTTAACACCTCCGTGGTGTACAAGAAAACCCTCTTTG |
| Nucleotide | TCGAGTTCACTGACCACCTGTTCAACATCGCCAAGCCGCGCCCACCTTGGATGGGCCTCCTGGGACCGACCATTCAA |
| sequence | GCTGAAGTGTACGACACCGTGGTGATCACCCTGAAGAACATGGCGTCCCACCCCGTGTCCCTGCATGCGGTCGGAGT |
| encoding | GTCCTACTGGAAGGCCTCCGAAGGAGCTGAGTACGACGACCAGACTAGCCAGCGGGAAAAGGAGGACGATAAAGTGT |
| BDD- | TCCCGGGCGGCTCGCATACTTACGTGTGGCAAGTCCTGAAGGAAAACGGACCTATGGCATCCGATCCTCTGTGCCTG |
| co6FVIII | ACTTACTCCTACCTTTCCCATGTGGACCTCGTGAAGGACCTGAACAGCGGGCTGATTGGTGCACTTCTCGTGTGCCG |
| (V1.0) | CGAAGGTTCGCTCGCTAAGGAAAAGACCCAGACCCTCCATAAGTTCATCCTTTTGTTCGCTGTGTTCGATGAAGGAA |
| (no XTEN) | AGTCATGGCATTCCGAAACTAAGAACTCGCTGATGCAGGACCGGGATGCCGCCTCAGCCCGCGCCTGGCCTAAAATG |
| | CATACAGTCAACGGATACGTGAATCGGTCACTGCCCGGGCTCATCGGTTGTCACAGAAAGTCCGTGTACTGGCACGT |
| | CATCGGCATGGGCACTACGCCTGAAGTGCACTCCATCTTCCTGGAAGGGCACACCTTCCTCGTGCGCAACCACCGCC |
| | AGGCCTCTCTGGAAATCTCCCCGATTACCTTTCTGACCGCCCAGACTCTGCTCATGGACCTGGGGCAGTTCCTTCTC |
| | TTCTGCCACATCTCCAGCCATCAGCACGACGGAATGGAGGCCTACGTGAAGGTGGACTCATGCCCGGAAGAACCTCA |
| | GTTGCGGATGAAGAACAACGAGGAGGCCGAGGACTATGACGACGATTTGACTGACTCCGAGATGGACGTCGTGCGGT |
| | TCGATGACGACAACAGCCCCAGCTTCATCCAGATTCGCAGCGTGGCCAAGAAGCACCCCAAAACCTGGGTGCACTAC |
| | ATCGCGGCCGAGGAAGAAGATTGGGACTACGCCCCGTTGGTGCTGGCACCCGATGACCGGTCGTACAAGTCCCAGTA |
| | TCTGAACAATGGTCCGCAGCGGATTGGCAGAAAGTACAAGAAAGTGCGGTTCATGGCGTACACTGACGAAACGTTTA |
| | AGACCCGGGAGGCCATTCAACATGAGAGCGGCATTCTGGGACCACTGCTGTACGGAGAGGTCGGCGATACCCTGCTC |
| | ATCATCTTCAAAAACCAGGCCTCCCGGCCTTACAACATCTACCCTCACGGAATCACCGACGTGCGGCCACTCTACTC |
| | GCGGCGCCTGCCGAAGGGCGTCAAGCACCTGAAAGACTTCCCTATCCTGCCGGGCGAAATCTTCAAGTATAAGTGGA |
| | CCGTCACCGTGGAGGACGGGCCCACCAAGAGCGATCCTAGGTGTCTGACTCGGTACTACTCCAGCTTCGTGAACATG |
| | GAACGGGACCTGGCATCGGGACTCATTGGACCGCTGCTGATCTGCTACAAAGAGTCGGTGGATCAACGCGGCAACCA |
| | GATCATGTCCGACAAGCGCAACGTGATCCTGTTCTCCGTGTTTGATGAAAACAGATCCTGGTACCTCACTGAAAACA |
| | TCCAGAGGTTCCTCCCAAACCCCGCAGGAGTGCAACTGGAGGACCCTGAGTTTCAGGCCTCGAATATCATGCACTCG |
| | ATTAACGGTTACGTGTTCGACTCGCTGCAGCTGAGCGTGTGCCTCCATGAAGTCGCTTACTGGTACATTCTGTCCAT |
| | CGGCGCCCAGACTGACTTCCTGAGCGTGTTCTTTTCCGGTTACACCTTTAAGCACAAGATGGTGTACGAAGATACCC |
| | TGACCCTGTTCCCTTTCTCCGGCGAAACGGTGTTCATGTCGATGGAGAACCCGGGTCTGTGGATTCTGGGATGCCAC |
| | AACAGCGACTTTCGGAACCGCGGAATGACTGCCCTGCTGAAGGTGTCCTCATGCGACAAGAACACCGGAGACTACTA |
| | CGAGGACTCCTACGAGGATATCTCAGCCTACCTCCTGTCCAAGAACAACGCGATCGAGCCGCGCAGCTTCAGCCAGA |
| | ACCCGCCTGTGCTGAAGAGGCACCAGCGAGAAATTACCCGGACCACCCTCCAATCGGATCAGGAGGAAATCGACTAC |
| | GACGACACCATCTCGGTGGAAATGAAGAAGGAAGATTTCGATATCTACGACGAGGACGAAAATCAGTCCCCTCGCTC |
| | ATTCCAAAAGAAAACTAGACACTACTTTATCGCCGCGGTGGAAAGACTGTGGGACTATGGAATGTCATCCAGCCCTC |
| | ACGTCCTTCGGAACCGGGCCCAGAGCGGATCGGTGCCTCAGTTCAAGAAAGTGGTGTTCCAGGAGTTCACCGACGGC |
| | AGCTTCACCCAGCCGCTGTACCGGGGAGAACTGAACGAACACCTGGGCCTGCTCGGTCCCTACATCCGCGCGGAAGT |
| | GGAGGATAACATCATGGTGACCTTCCGTAACCAAGCATCCAGACCTTACTCCTTCTATTCCTCCCTGATCTCATACG |
| | AGGAGGACCAGCGCCAAGGCGCCGAGCCCCGCAAGAACTTCGTCAAGCCCAACGAGACTAAGACCTACTTCTGGAAG |
| | GTCCAACACCATATGGCCCCGACCAAGGATGAGTTTGACTGCAAGGCCTGGGCCTACTTCTCCGACGTGGACCTTGA |
| | GAAGGATGTCCATTCCGGCCTGATCGGGCCGCTGCTCGTGTGTCACACCAACACCCTGAACCCAGCGCATGGACGCC |
| | AGGTCACCGTCCAGGAGTTTGCTCTGTTCTTCACCATTTTTGACGAAACTAAGTCCTGGTACTTCACCGAGAATATG |
| | GAGCGAAACTGTAGAGCGCCCTGCAATATCCAGATGGAAGATCCGACTTTCAAGGAGAACTATAGATTCCACGCCAT |
| | CAACGGGTACATCATGGATACTCTGCCGGGGCTGGTCATGGCCCAGGATCAGAGGATTCGGTGGTACTTGCTGTCAA |
| | TGGGATCGAACGAAAACATTCACTCCATTCACTTCTCCGGTCACGTGTTCACTGTGCGCAAGAAGGAGGAGTACAAG |
| | ATGGCGCTGTACAATCTGTACCCCGGGGTGTTCGAAACTGTGGAGATGCTGCCGTCCAAGGCCGGCATCTGGAGAGT |
| | GGAGTGCCTGATCGGAGAGCACCTCCACGCGGGGATGTCCACCCTCTTCCTGGTGTACTCGAATAAGTGCCAGACCC |
| | CGCTGGGCATGGCCTCGGGCCACATCAGAGACTTCCAGATCACAGCAAGCGGACAATACGGCCAATGGGCGCCGAAG |
| | CTGGCCCGCTTGCACTACTCCGGATCGATCAACGCATGGTCCACCAAGGAACCGTTCTCGTGGATTAAGGTGGACCT |
| | CCTGGCCCCTATGATTATCCACGGAATTAAGACCCAGGGCGCCAGGCAGAAGTTCTCCTCCCTGTACATCTCGCAAT |
| | TCATCATCATGTACAGCCTGGACGGGAAGAAGTGGCAGACTTACAGGGGAAACTCCACCGGCACCCTGATGGTCTTT |
| | TTCGGCAACGTGGATTCCTCCGGCATTAAGCACAACATCTTCAACCCACCGATCATAGCCAGATATATTAGGCTCCA |
| | CCCCACTCACTACTCAATCCGCTCAACTCTTCGGATGGAACTCATGGGGTGCGACCTGAACTCCTGCTCCATGCCGT |
| | TGGGGATGGAATCAAAGGCTATTAGCGACGCCCAGATCACCGCGAGCTCCTACTTCACTAACATGTTCGCCACCTGG |
| | AGCCCCTCCAAGGCCAGGCTGCACTTGCAGGGACGGTCAAATGCCTGGCGGCCGCAAGTGAACAATCCGAAGGAATG |
| | GCTTCAAGTGGATTTCCAAAAGACCATGAAAGTGACCGGAGTCACCACCCAGGGAGTGAAGTCCCTTCTGACCTCGA |
| | TGTATGTGAAGGAGTTCCTGATTAGCAGCAGCCAGGACGGGCACCAGTGGACCCTGTTCTTCCAAAACGGAAAGGTC |
| | AAGGTGTTCCAGGGGAACCAGGACTCGTTCACACCCGTGGTGAACTCCCTGGACCCCCCACTGCTGACGCGGTACTT |
| | GAGGATTCATCCTCAGTCCTGGGTCCATCAGATTGCATTGCGAATGGAAGTCCTGGGCTGCGAGGCCCAGGACCTGT |
| | ACTGA |
| SEQ ID | GCCACCCGCCGGTATTACTTAGGTGCTGTGGAACTGAGCTGGGACTACATGCAGTCCGACCTGGGAGAACTGCCGGT |
| NO: 22 | GGACGCGAGATTCCCACCTAGAGTCCCGAAGTCCTTCCCATTCAACACCTCCGTGGTCTACAAAAAGACCCTGTTCG |
| Nucleotide | TGGAGTTCACTGACCACCTTTTCAATATTGCCAAGCCGCGCCCCCCCTGGATGGGCCTGCTTGGTCCTACGATCCAA |
| sequence | GCAGAGGTCTACGACACCGTGGTCATCACACTGAAGAACATGGCCTCACACCCCGTGTCGCTGCATGCTGTGGGAGT |
| encoding | GTCCTACTGGAAGGCCTCAGAGGGTGCCGAATATGATGACCAGACCAGCCAGAGGGAAAAGGAGGATGACAAAGTGT |
| coBDDFVIII | TCCCGGGTGGCAGCCACACTTACGTGTGGCAAGTGCTGAAGGAAAACGGGCCTATGGCGTCGGACCCCCTATGCCTG |
| (V2.0) | ACCTACTCCTACCTGTCCCATGTGGACCTTGTGAAGGATCTCAACTCGGGACTGATCGGCGCCCTCTTGGTGTGCAG |
| (no XTEN) | AGAAGGCAGCCTGGCGAAGGAAAAGACTCAGACCCTGCACAAGTTCATTCTGTTGTTTGCTGTGTTCGATGAAGGAA |
| | AGTCCTGGCACTCAGAAACCAAGAACTCGCTGATGCAGGATAGAGATGCGGCCTCGGCCAGAGCCTGGCCTAAAATG |
| | CACACCGTCAACGGATATGTGAACAGGTCGCTCCCTGGCCTCATCGGCTGCCACAGAAAGTCCGTGTATTGGCATGT |
| | GATCGGCATGGGTACTACTCCGGAAGTGCATAGTATCTTTCTGGAGGGCCATACCTTCTTGGTGCGCAACCACAGAC |
| | AGGCCTCGCTGGAAATCTCGCCTATCACTTTCTTGACTGCGCAGACCCTCCTTATGGACCTTGGACAGTTCCTGCTG |
| | TTCTGTCACATCAGCTCCCATCAGCATGATGGGATGGAGGCCTATGTCAAAGTGGACTCCTGCCCTGAGGAGCCACA |
| | GCTCCGGATGAAGAACAATGAGGAAGCGGAGGATTACGACGACGACCTGACTGACAGCGAAATGGACGTCGTGCGAT |
| | TCGATGACGACAACAGCCCGTCCTTCATCCAAATTAGATCAGTGGCGAAGAAGCACCCCAAGACCTGGGTGCACTAC |
| | ATTGCCGCCGAGGAAGAGGACTGGGACTACGCGCCGCTGGTGCTGGCGCCAGACGACAGGAGCTACAAGTCCCAGTA |
| | CCTCAACAACGGGCCGCAGCGCATTGGCAGGAAGTACAAGAAAGTCCGCTTCATGGCCTACACTGATGAAACCTTCA |
| | AGACGAGGGAAGCCATCCAGCACGAGTCAGGCATCCTGGGACCGCTCCTTTACGGCGAAGTCGGGGATACCCTGCTC |
| | ATCATTTTCAAGAACCAGGCATCGCGGCCCTACAACATCTACCCTCACGGGATCACAGACGTGCGCCCGCTCTACTC |
| | CCGCCGGCTGCCCAAGGGAGTGAAGCACCTGAAGGATTTTCCCATCCTGCCGGGAGAAATCTTCAAGTACAAGTGGA |
| | CCGTGACTGTGGAAGATGGCCCTACCAAGTCGGACCCTCGCTGTCTGACCCGGTACTATTCCTCGTTTGTGAACATG |
| | GAGCGCGACCTGGCCTCGGGGCTGATTGGTCCGCTGCTGATCTGCTACAAGGAGTCCGTGGACCAGCGCGGGAACCA |
| | GATCATGTCCGACAAGCGCAACGTGATCCTGTTCTCTGTCTTTGATGAAAACAGATCGTGGTACTTGACTGAGAATA |
| | TCCAGCGGTTCCTGCCCAACCCAGCGGGAGTGCAACTGGAGGACCCGGAGTTCCAGGCCTCAAACATTATGCACTCT |
| | ATCAACGGCTATGTGTTCGACTCGCTCCAACTGAGCGTGTGCCTGCATGAAGTGGCATACTGGTACATTCTGTCCAT |
| | CGGAGCCCAGACCGACTTCCTGTCCGTGTTCTTCTCCGGATACACCTTCAAGCATAAGATGGTGTACGAGGACACTC |
| | TGACCCTCTTCCCATTTTCCGGAGAAACTGTGTTCATGTCAATGGAAAACCCGGGCTTGTGGATTCTGGGTTGCCAT |
| | AACTCGGACTTCCGGAATAGAGGGATGACCGCCCTGCTGAAAGTGTCCAGCTGTGACAAGAATACCGGCGATTACTA |
| | CGAGGACAGCTATGAGGACATCTCCGCTTATCTGCTGTCCAAGAACAACGCCATTGAACCCAGGTCCTTCTCCCAAA |
| | ACGGTGCACCGGCCTCATCCCCCCCCGTGCTGAAGCGGCATCAAAGAGAGATCACCAGGACCACTCTCCAGTCCGAT |
| | CAGGAAGAAATTGACTACGACGATACTATCAGCGTGGAGATGAAGAAGGAGGACTTCGACATCTACGATGAGGATGA |
| | GAACCAGTCCCCTCGGAGCTTTCAGAAGAAAACCCGCCACTACTTCATCGCTGCCGTGGAGCGGCTGTGGGATTACG |
| | GGATGTCCAGCTCACCGCATGTGCTGCGGAATAGAGCGCAGTCAGGATCGGTGCCCCAGTTCAAGAAGGTCGTGTTC |
| | CAAGAGTTCACCGACGGGTCCTTCACTCAACCCCTGTACCGGGGCGAACTCAACGAACACCTGGGACTGCTTGGGCC |
| | GTATATCAGGGCAGAAGTGGAAGATAACATCATGGTCACCTTCCGCAACCAGGCCTCCCGGCCGTACAGCTTCTACT |
| | CTTCACTGATCTCCTACGAGGAAGATCAGCGGCAGGGAGCCGAGCCCCGGAAGAACTTCGTCAAGCCTAACGAAACT |
| | AAGACCTACTTTTGGAAGGTCCAGCATCACATGGCCCCGACCAAAGACGAGTTCGACTGTAAAGCCTGGGCCTACTT |
| | CTCCGATGTGGACCTGGAGAAGGACGTGCACTCGGGACTCATTGGCCCGCTCCTTGTGTGCCATACTAATACCCTGA |
| | ACCCTGCTCACGGTCGCCAAGTCACAGTGCAGGAGTTCGCCCTCTTCTTCACCATCTTCGATGAAACAAAGTCCTGG |
| | TACTTTACTGAGAACATGGAACGCAATTGCAGGGCACCCTGCAACATCCAGATGGAAGATCCCACCTTCAAGGAAAA |
| | CTACCGGTTTCATGCCATTAACGGCTACATAATGGACACGTTGCCAGGACTGGTCATGGCCCAGGACCAGAGAATCC |
| | GGTGGTATCTGCTCTCCATGGGCTCCAACGAAAACATTCACAGCATTCATTTTTCCGGCCATGTGTTCACCGTCCGG |
| | AAGAAGGAAGAGTACAAGATGGCTCTGTACAACCTCTACCCTGGAGTGTTCGAGACTGTGGAAATGCTGCCTAGCAA |
| | GGCCGGCATTTGGAGAGTGGAATGCCTGATCGGAGAGCATTTGCACGCCGGAATGTCCACCCTGTTTCTTGTGTACT |
| | CCAACAAGTGCCAGACCCCGCTGGGAATGGCCTCAGGTCATATTAGGGATTTCCAGATCACTGCTTCGGGGCAGTAC |
| | GGGCAGTGGGCACCTAAGTTGGCCCGGCTGCACTACTCTGGCTCCATCAATGCCTGGTCCACCAAGGAACCCTTCTC |
| | CTGGATTAAGGTGGACCTCCTGGCCCCAATGATTATTCACGGTATTAAGACCCAGGGTGCCCGACAGAAGTTCTCCT |
| | CACTCTACATCTCGCAATTCATCATAATGTACAGCCTGGATGGGAAGAAGTGGCAGACCTACCGGGGAAACTCCACT |
| | GGAACGCTCATGGTGTTTTTCGGCAACGTGGACTCCTCCGGCATTAAGCACAACATCTTCAACCCTCCGATCATTGC |
| | TCGGTACATCCGGCTGCACCCAACTCACTACAGCATCCGGTCCACCCTGCGGATGGAACTGATGGGTTGTGACCTGA |
| | ACTCCTGCTCCATGCCCCTTGGGATGGAATCCAAGGCCATTAGCGATGCACAGATCACCGCCTCTTCATACTTCACC |
| | AACATGTTCGCGACCTGGTCCCCGTCGAAGGCCCGCCTGCACCTCCAAGGTCGCTCCAATGCGTGGCGGCCTCAAGT |
| | GAACAACCCCAAGGAGTGGCTCCAGGTCGACTTCCAAAAGACCATGAAGGTCACCGGAGTGACCACCCAGGGCGTGA |
| | AGTCCCTGCTGACCTCTATGTACGTTAAGGAGTTCCTCATCTCCTCAAGCCAAGACGGACATCAGTGGACCCTGTTC |
| | TTCCAAAACGGAAAAGTCAAAGTATTCCAGGGCAACCAGGACTCCTTCACCCCTGTGGTCAACAGCCTGGACCCCCC |
| | ATTGCTGACCCGCTACCTCCGCATCCACCCCCAAAGCTGGGTCCACCAGATCGCACTGCGCATGGAGGTCCTTGGAT |
| | GCGAAGCCCAAGATCTGTACTAA |
| SEQ ID | ATGCAGATTGAGCTGTCCACTTGTTTCTTCCTGTGCCTCCTGCGCTTCTGTTTCTCCGCCACTCGCCGGTACTACCT |
| NO: 23 | TGGAGCCGTGGAGCTTTCATGGGACTACATGCAGAGCGACCTGGGCGAACTCCCCGTGGATGCCAGATTCCCCCCCC |
| V1.0 | GCGTGCCAAAGTCCTTCCCCTTTAACACCTCCGTGGTGTACAAGAAAACCCTCTTTGTCGAGTTCACTGACCACCTG |
| Expression | TTCAACATCGCCAAGCCGCGCCCACCTTGGATGGGCCTCCTGGGACCGACCATTCAAGCTGAAGTGTACGACACCGT |
| cassette | GGTGATCACCCTGAAGAACATGGCGTCCCACCCCGTGTCCCTGCATGCGGTCGGAGTGTCCTACTGGAAGGCCTCCG |
| TTP- | AAGGAGCTGAGTACGACGACCAGACTAGCCAGCGGGAAAAGGAGGACGATAAAGTGTTCCCGGGCGGCTCGCATACT |
| Intron- | TACGTGTGGCAAGTCCTGAAGGAAAACGGACCTATGGCATCCGATCCTCTGTGCCTGACTTACTCCTACCTTTCCCA |
| BDDFVIIIc | TGTGGACCTCGTGAAGGACCTGAACAGCGGGCTGATTGGTGCACTTCTCGTGTGCCGCGAAGGTTCGCTCGCTAAGG |
| o6XTEN | AAAAGACCCAGACCCTCCATAAGTTCATCCTTTTGTTCGCTGTGTTCGATGAAGGAAAGTCATGGCATTCCGAAACT |
| (V1.0)- | AAGAACTCGCTGATGCAGGACCGGGATGCCGCCTCAGCCCGCGCCTGGCCTAAAATGCATACAGTCAACGGATACGT |
| WPRE- | GAATCGGTCACTGCCCGGGCTCATCGGTTGTCACAGAAAGTCCGTGTACTGGCACGTCATCGGCATGGGCACTACGC |
| bGHPolyA | CTGAAGTGCACTCCATCTTCCTGGAAGGGCACACCTTCCTCGTGCGCAACCACCGCCAGGCCTCTCTGGAAATCTCC |
| expression | CCGATTACCTTTCTGACCGCCCAGACTCTGCTCATGGACCTGGGGCAGTTCCTTCTCTTCTGCCACATCTCCAGCCA |
| cassette | TCAGCACGACGGAATGGAGGCCTACGTGAAGGTGGACTCATGCCCGGAAGAACCTCAGTTGCGGATGAAGAACAACG |
| | AGGAGGCCGAGGACTATGACGACGATTTGACTGACTCCGAGATGGACGTCGTGCGGTTCGATGACGACAACAGCCCC |
| | AGCTTCATCCAGATTCGCAGCGTGGCCAAGAAGCACCCCAAAACCTGGGTGCACTACATCGCGGCCGAGGAAGAAGA |
| | TTGGGACTACGCCCCGTTGGTGCTGGCACCCGATGACCGGTCGTACAAGTCCCAGTATCTGAACAATGGTCCGCAGC |
| | GGATTGGCAGAAAGTACAAGAAAGTGCGGTTCATGGCGTACACTGACGAAACGTTTAAGACCCGGGAGGCCATTCAA |
| | CATGAGAGCGGCATTCTGGGACCACTGCTGTACGGAGAGGTCGGCGATACCCTGCTCATCATCTTCAAAAACCAGGC |
| | CTCCCGGCCTTACAACATCTACCCTCACGGAATCACCGACGTGCGGCCACTCTACTCGCGGCGCCTGCCGAAGGGCG |
| | TCAAGCACCTGAAAGACTTCCCTATCCTGCCGGGCGAAATCTTCAAGTATAAGTGGACCGTCACCGTGGAGGACGGG |
| | CCCACCAAGAGCGATCCTAGGTUCTGACTCGGTACTACTCCAGCTTCGTGAACATGGAACGGGACCTGGCATCGGG |
| | ACTCATTGGACCGCTGCTGATCTGCTACAAAGAGTCGGTGGATCAACGCGGCAACCAGATCATGTCCGACAAGCGCA |
| | ACGTGATCCTGTTCTCCGTGTTTGATGAAAACAGATCCTGGTACCTCACTGAAAACATCCAGAGGTTCCTCCCAAAC |
| | CCCGCAGGAGTGCAACTGGAGGACCCTGAGTTTCAGGCCTCGAATATCATGCACTCGATTAACGGTTACGTGTTCGA |
| | CTCGCTGCAACTGAGCGTGTGCCTCCATGAAGTCGCTTACTGGTACATTCTGTCCATCGGCGCCCAGACTGACTTCC |
| | TGAGCGTGTTCTTTTCCGGTTACACCTTTAAGCACAAGATGGTGTACGAAGATACCCTGACCCTGTTCCCTTTCTCC |
| | GGCGAAACGGTGTTCATGTCGATGGAGAACCCGGGTCTGTGGATTCTGGGATGCCACAACAGCGACTTTCGGAACCG |
| | CGGAATGACTGCCCTGCTGAAGGTGTCCTCATGCGACAAGAACACCGGAGACTACTACGAGGACTCCTACGAGGATA |
| | TCTCAGCCTACCTCCTGTCCAAGAACAACGCGATCGAGCCGCGCAGCTTCAGCCAGAACGGCGCGCCAACATCAGAG |
| | AGCGCCACCCCTGAAAGTGGTCCCGGGAGCGAGCCAGCCACATCTGGGTCGGAAACGCCAGGCACAAGTGAGTCTGC |
| | AACTCCCGAGTCCGGACCTGGCTCCGAGCCTGCCACTAGCGGCTCCGAGACTCCGGGAACTTCCGAGAGCGCTACAC |
| | CAGAAAGCGGACCCGGAACCAGTACCGAACCTAGCGAGGGCTCTGCTCCGGGCAGCCCAGCCGGCTCTCCTACATCC |
| | ACGGAGGAGGGCACTTCCGAATCCGCCACCCCGGAGTCAGGGCCAGGATCTGAACCCGCTACCTCAGGCAGTGAGAC |
| | GCCAGGAACGAGCGAGTCCGCTACACCGGAGAGTGGGCCAGGGAGCCCTGCTGGATCTCCTACGTCCACTGAGGAAG |
| | GGTCACCAGCGGGCTCGCCCACCAGCACTGAAGAAGGTGCCTCGAGCCCGCCTGTGCTGAAGAGGCACCAGCGAGAA |
| | ATTACCCGGACCACCCTCCAATCGGATCAGGAGGAAATCGACTACGACGACACCATCTCGGTGGAAATGAAGAAGGA |
| | AGATTTCGATATCTACGACGAGGACGAAAATCAGTCCCCTCGCTCATTCCAAAAGAAAACTAGACACTACTTTATCG |
| | CCGCGGTGGAAAGACTGTGGGACTATGGAATGTCATCCAGCCCTCACGTCCTTCGGAACCGGGCCCAGAGCGGATCG |
| | GTGCCTCAGTTCAAGAAAGTGGTGTTCCAGGAGTTCACCGACGGCAGCTTCACCCAGCCGCTGTACCGGGGAGAACT |
| | GAACGAACACCTGGGCCTGCTCGGTCCCTACATCCGCGCGGAAGTGGAGGATAACATCATGGTGACCTTCCGTAACC |
| | AAGCATCCAGACCTTACTCCTTCTATTCCTCCCTGATCTCATACGAGGAGGACCAGCGCCAAGGCGCCGAGCCCCGC |
| | AAGAACTTCGTCAAGCCCAACGAGACTAAGACCTACTTCTGGAAGGTCCAACACCATATGGCCCCGACCAAGGATGA |
| | GTTTGACTGCAAGGCCTGGGCCTACTTCTCCGACGTGGACCTTGAGAAGGATGTCCATTCCGGCCTGATCGGGCCGC |
| | TGCTCGTGTGTCACACCAACACCCTGAACCCAGCGCATGGACGCCAGGTCACCGTCCAGGAGTTTGCTCTGTTCTTC |
| | ACCATTTTTGACGAAACTAAGTCCTGGTACTTCACCGAGAATATGGAGCGAAACTGTAGAGCGCCCTGCAATATCCA |
| | GATGGAAGATCCGACTTTCAAGGAGAACTATAGATTCCACGCCATCAACGGGTACATCATGGATACTCTGCCGGGGC |
| | TGGTCATGGCCCAGGATCAGAGGATTCGGTGGTACTTGCTGTCAATGGGATCGAACGAAAACATTCACTCCATTCAC |
| | TTCTCCGGTCACGTGTTCACTGTGCGCAAGAAGGAGGAGTACAAGATGGCGCTGTACAATCTGTACCCCGGGGTGTT |
| | CGAAACTUGGAGATGCTGCCGTCCAAGGCCGGCATCTGGAGAGTGGAGTGCCTGATCGGAGAGCACCTCCACGCGG |
| | GGATGTCCACCCTCTTCCTGGTGTACTCGAATAAGTGCCAGACCCCGCTGGGCATGGCCTCGGGCCACATCAGAGAC |
| | TTCCAGATCACAGCAAGCGGACAATACGGCCAATGGGCGCCGAAGCTGGCCCGCTTGCACTACTCCGGATCGATCAA |
| | CGCATGGTCCACCAAGGAACCGTTCTCGTGGATTAAGGTGGACCTCCTGGCCCCTATGATTATCCACGGAATTAAGA |
| | CCCAGGGCGCCAGGCAGAAGTTCTCCTCCCTGTACATCTCGCAATTCATCATCATGTACAGCCTGGACGGGAAGAAG |
| | TGGCAGACTTACAGGGGAAACTCCACCGGCACCCTGATGGTCTTTTTCGGCAACGTGGATTCCTCCGGCATTAAGCA |
| | CAACATCTTCAACCCACCGATCATAGCCAGATATATTAGGCTCCACCCCACTCACTACTCAATCCGCTCAACTCTTC |
| | GGATGGAACTCATGGGGTGCGACCTGAACTCCTGCTCCATGCCGTTGGGGATGGAATCAAAGGCTATTAGCGACGCC |
| | CAGATCACCGCGAGCTCCTACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGATTTCCAAAAGACCATGAAAG |
| | TGACCGGAGTCACCACCCAGGGAGTGAAGTCCCTTCTGACCTCGATGTATGTGAAGGAGTTCCTGATTAGCAGCAGC |
| | CAGGACGGGCACCAGTGGACCCTGTTCTTCCAAAACGGAAAGGTCAAGGTGTTCCAGGGGAACCAGGACTCGTTCAC |
| | ACCCGTGGTGAACTCCCTGGACCCCCCACTGCTGACGCGGTACTTGAGGATTCATCCTCAGTCCTGGGTCCATCAGA |
| | TTGCATTGCGAATGGAAGTCCTGGGCTGCGAGGCCCAGGACCTGTACTGA |

1. A recombinant bacmid, comprising:
(i) a variant of a baculovirus gene required for baculovirus replication, wherein the variant gene exhibits reduced expression of its encoded protein;
(ii) a bacterial origin of replication (ori); and
(iii) at least one integration site for integration of a heterologous DNA sequence comprising a transgene.

2. (canceled)

3. The bacmid of claim 1, wherein the baculovirus gene is selected from the group consisting of VP80, VP39, GP41, P333, VP1-54, VLF-1, and PP78/83.

4. The bacmid of claim 1, wherein the baculovirus gene is VP80.

5-10. (canceled)

11. The bacmid of claim 1, further comprising a Rep protein.

12. A recombinant baculovirus expression vector (rBEV) generated by site specific integration of a heterologous DNA sequence into the integration site of the bacmid of claim 1.

13. The rBEV of claim 12, wherein the heterologous nucleic acid sequence comprises a transgene flanked by Inverted Terminal Repeats (ITRs).

14. The rBEV of claim 12, wherein the heterologous nucleic acid is expressed as closed ended DNA (ceDNA).

15. A baculovirus expression system comprising
(i) the rBEV of claim 12; and
(ii) a source of functional protein wherein the functional protein is capable of complementing the variant essential gene and wherein the functional protein is provided in trans to the rBEV.

16. The baculovirus expression system of claim 15, wherein the functional protein is provided as a separate expression vector which expresses the functional protein in trans.

17. (canceled)

18. The baculovirus expression system of claim 15, wherein the functional protein is provided by an insect cell, wherein the insect cell is optionally a Sf9, Sf21, S2, Trichoplusia ni, E4a, or BTI-TN-5B1-4 cell.

19. A method of propagating a baculovirus expression vector in an insect cell the method comprising:
(a) transfecting the insect cell with the recombinant baculovirus expression vector (rBEV) of claim 12;
(b) providing a functional protein capable of complementing the variant baculovirus gene wherein the functional protein is provided in trans to the rBEV; and
(c) culturing the insect cell, thereby propagating the baculovirus expression system vector.

20. (canceled)

21. The method of claim 19, wherein the functional protein is provided by transfecting the insect cell with a separate expression vector which stably integrates and expresses the functional protein in trans.

22. (canceled)

23. The method of claim 19, wherein the functional protein is expressed in the cell under the control of an inducible or transactivating promoter, wherein the inducible promoter is optionally the Autographa californica nucleopolyhedrovirus (AcMNPV) 39K promoter.

24. (canceled)

25. A method of producing a heterologous DNA sequence comprising a transgene,
(a) propagating a recombinant baculovirus expression vector (rBEV) according to the method of claim 19;
(b) harvesting the rBEV;
(c) infecting an insect cell to express a heterologous DNA sequence; and
(d) purifying the heterologous DNA sequence from the insect cell.

26. (canceled)

27. The method of claim 25, wherein the heterologous DNA sequence comprises the transgene flanked by Inverted Terminal Repeats (ITRs).

28. The method of claim 27, wherein the ITRs are derived from a parovirus, wherein the parovirus is optionally B19, GPV, HBoV1, or AAV.

29. (canceled)

30. The method of claim 27, wherein the heterologous nucleic acid molecule is expressed as closed ended DNA (ceDNA).

31. The method of claim 27, wherein the heterologous nucleic acid molecule comprises the nucleotide sequence of SEQ ID NO: 15, and wherein the nucleotide sequence encodes a polypeptide with Factor VIII activity.

32. The method of claim 27, wherein the heterologous nucleic acid molecule comprises a genetic cassette comprising the nucleotide sequence of SEQ ID NO: 14.

33. A heterologous DNA sequence comprising a transgene encoding a therapeutic protein, wherein the heterologous DNA sequence is produced by the method of claim 25.
